SEQUENCE LISTING (13 sequences)

SEQ ID NO: 1 - protein, 451 aa, Artificial Sequence
  RheCD4-Ig
MDWTWRILFLVAAATGTHAKKVVLGKKGDTVELTCNASQKKNTQFHWKNSNQIKILGNQGSFLTKGPSKLSDRADSRKSLWDQGCFSMIIKNLKIEDSDTYICEVENKKEEVELLVFGLTANSDTHLLEGQSLTLTLESPPGSSPSVKCRSPGGKNIQGGRTISVPQLERQDSGTWTCTVSQDQKTVEFKIDIVVLAFQKASSTGLPCRSTCPPCPAELLGGPSVFLFPPKPKDTLMISRTPEVTCVVVDVSQEEPDVKFNWYVDGVEVHNAQTKPREEQFNSTYRVVSVLTVTHQDWLNGKEYTCKVSNKALPAPRQKTVSKTKGQPREPQVYTLPPPREELTKNQVSLTCLVKGFYPSDIVVEWASNGQPENTYKTTPPVLDSDGSYFLYSKLTVDKSRWQQGNTFSCSVMHEALHNHYTQKSLSVSPGKGGGGGDYYDYDGGYYYDMD

SEQ ID NO: 2 - DNA, 1377 bp, Artificial Sequence
  RheCD4-Ig
ggatccgccaccatggactggacttggagaatcctgttcctggtcgccgctgctaccgggactcacgctaaaaaggtcgtgctgggcaaaaagggagatacagtggagctgacctgcaacgcctcccagaagaagaatacacagttccactggaagaacagcaatcagatcaagatcctgggcaatcagggctcctttctgaccaagggaccaagcaagctgtccgacagggcagattctagaaagagcctgtgggaccagggctgcttctctatgatcatcaagaacctgaagatcgaggacagcgatacctacatctgtgaggtggagaataagaaggaggaggtggagctgctggtgtttggcctgacagccaactccgatacccacctgctggagggacagtccctgaccctgacactggagtctccccctggcagctccccaagcgtgaagtgcaggtcccctggcggcaagaacatccagggcggccggacaatcagcgtgcctcagctggagcgccaggactctggcacctggacatgtaccgtgagccaggaccagaagaccgtggagttcaagatcgatatcgtggtgctggcctttcagaaggcctctagcacaggcctgccatgcaggtctacctgcccaccctgtccagcagagctgctgggcggcccaagcgtgttcctgtttcctccaaagcctaaggacaccctgatgatctccagaacaccagaggtgacctgcgtggtggtggacgtgtctcaggaggagccagatgtgaagttcaactggtacgtggatggcgtggaggtgcacaatgcccagacaaagcccagggaggagcagtttaattctacctacagagtggtgagcgtgctgacagtgacccaccaggattggctgaacggcaaggagtatacatgcaaggtgagcaataaggccctgccagcccccaggcagaagacagtgtccaagaccaagggccagcctcgggagccacaggtgtataccctgccccctccacgcgaggagctgacaaagaaccaggtgagcctgacctgtctggtgaagggcttctacccctccgacatcgtggtggagtgggcctctaacggccagcctgagaatacatataagaccacaccccctgtgctggactccgatggctcttacttcctgtatagcaagctgacagtggataagtcccgctggcagcagggcaacaccttttcctgttctgtgatgcacgaggccctgcacaatcactatacccagaagagcctgtccgtgtctcctggcaagggcggaggaggcggagactattacgactatgacggcggctactactatgatatggattgataactcgag

SEQ ID NO: 3 - protein, 453 aa, Artificial Sequence
  human eCD4-Ig
MDWTWRILFLVAAATGTHAKKVVLGKKGDTVELTCTASQKKSIQFHWKNSNQIKILGNAGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAAADPEPKSCDKTHTCPPCPAPELLGGPSVFLFPPKPPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGDYADYDGGYYYDMD

SEQ ID NO: 4 - DNA, 1383 bp, Artificial Sequence
  human eCD4-Ig
ggatccgccaccatggactggacttggaggattctgtttctggtcgccgccgctactggaacccacgctaaaaaggtcgtgctgggcaaaaagggagacacagtggagctgacctgcacagcctcccagaagaagtctatccagttccactggaagaactccaatcagatcaagatcctgggcaacgccggctcctttctgaccaagggcccatctaagctgaatgacagagccgatagcaggagatccctgtgggaccagggcaacttccccctgatcatcaagaatctgaagatcgaggactctgatacctacatctgcgaggtggaggaccagaaggaggaggtgcagctgctggtgtttggcctgacagccaacagcgatacccacctgctgcagggacagagcctgaccctgacactggagagccctcccggcagctccccaagcgtgcagtgtaggagccccaggggcaagaacatccagggcggcaagaccctgtctgtgagccagctggagctgcaggacagcggcacctggacatgcaccgtgctgcagaatcagaagaaggtggagttcaagatcgatatcgtggtgctggcagcagccgaccccgagcctaagagctgtgataagacacacacctgcccaccctgtccagcaccagagctgctgggcggcccttccgtgttcctgtttcctccaaagccccctaaggacacactgatgatctctaggacacccgaggtgacctgcgtggtggtggacgtgagccacgaggaccccgaggtgaagtttaactggtacgtggatggcgtggaggtgcacaatgccaagaccaagccaagggaggagcagtacaactctacatatagagtggtgagcgtgctgaccgtgctgcaccaggactggctgaacggcaaggagtataagtgcaaggtgtccaataaggccctgcctgccccaatcgagaagacaatctctaaggcaaagggacagccccgggagcctcaggtgtacaccctgccacccagccgcgacgagctgacaaagaaccaggtgtccctgacctgtctggtgaagggcttctatccttccgatatcgccgtggagtgggagtctaatggccagccagagaacaattacaagaccacacctccagtgctggactctgatggcagcttctttctgtattctaagctgacagtggataagagcaggtggcagcagggcaacgtgttttcctgttctgtgatgcacgaggccctgcacaatcactatacccagaagagcctgtccctgtctcccggcaagggcggcgggggcggagactatgctgactatgacggaggctattactatgacatggactgataactcgag

SEQ ID NO: 5 - protein, 394 aa, Artificial Sequence
  IgE TPST2
MDWTWILFLVAAATRVHSRLSVRRVLLAAGCALVLVLAVQLGQQVLECRAVLAGLRSPRGAMRPEQEELVMVGTNHVEYRYGKAMPLIFVGGVPRSGTTLMRAMLDAHPEVRCGEETRIIPRVLAMRQAWSKSGREKLRLDEAGVTDEVLDAAMQAFILEVIAKHGEPARVLCNKDPFTLKSSVYLSRLFPNSKFLLMVRDGRASVHSMITRKVTIAGFDLSSYRDCLTKWNKAIEVMYAQCMEVGKEKCLPVYYEQLVLHPRRSLKLILDFLGIAWSDAVLHHEDLIGKPGGVSLSKIERSTDQVIKPVNLEALSKWTGHIPGDVVRDMAQIAPMLAQLGYDPYANPPNYGNPDPFVINNTQRVLKGDYKTPANLKGYFQVNQNSTSSHLGSS

SEQ ID NO: 6 - DNA, 1206 bp, Artificial Sequence
  IgE TPST2
ggatccgccaccatggattggacctggattctgtttctggtcgctgccgctacaagggtgcattcaagactgtccgtgcgccgggtgctgctggccgctggatgcgcactggtgctggtgctggcagtgcagctgggacagcaggtgctggagtgtagggccgtgctggcaggcctgcggtcccctcgcggcgccatgcgcccagagcaggaggagctggtcatggtgggcaccaatcacgtggagtaccggtatggcaaggccatgccactgatcttcgtgggcggcgtgcctcggtctggaaccacactgatgagggcaatgctggacgcacacccagaggtgaggtgcggagaggagacaaggatcatcccccgcgtgctggcaatgagacaggcatggtccaagtctggcagggagaagctgagactggatgaggccggcgtgacagacgaggtgctggatgccgccatgcaggccttcatcctggaagtgatcgcaaagcacggagagccagcccgggtgctgtgcaataaggacccctttaccctgaagagctccgtgtacctgtcccgcctgttccccaactctaagtttctgctgatggtgagggatggcagagcctctgtgcacagcatgatcaccaggaaggtgacaatcgccggcttcgacctgtctagctacagagattgtctgacaaagtggaataaggccatcgaagtgatgtatgcccagtgcatggaagtgggcaaggagaagtgtctgcccgtgtactatgagcagctggtgctgcaccctaggagaagcctgaagctgatcctggactttctgggaatcgcatggtccgacgccgtgctgcaccacgaggatctgatcggcaagcccggcggcgtgagcctgtccaagatcgagaggtctaccgatcaggtcatcaagcctgtgaacctggaggccctgagcaagtggacaggccacatcccaggcgacgtggtgagagatatggcccagatcgcccctatgctggcccagctgggctacgacccatatgccaaccctcccaattacggcaacccagatccctttgtgatcaacaatacccagcgcgtgctgaagggcgactataagacacctgccaatctgaagggctatttccaggtcaaccagaactcaacatcctctcatctggggagcagttgataactcgag

SEQ ID NO: 7 - protein, 377 aa, Artificial Sequence
  TPST2
MRLSVRRVLLAAGCALVLVLAVQLGQQVLECRAVLAGLRSPRGAMRPEQEELVMVGTNHVEYRYGKAMPLIFVGGVPRSGTTLMRAMLDAHPEVRCGEETRIIPRVLAMRQAWSKSGREKLRLDEAGVTDEVLDAAMQAFILEVIAKHGEPARVLCNKDPFTLKSSVYLSRLFPNSKFLLMVRDGRASVHSMITRKVTIAGFDLSSYRDCLTKWNKAIEVMYAQCMEVGKEKCLPVYYEQLVLHPRRSLKLILDFLGIAWSDAVLHHEDLIGKPGGVSLSKIERSTDQVIKPVNLEALSKWTGHIPGDVVRDMAQIAPMLAQLGYDPYANPPNYGNPDPFVINNTQRVLKGDYKTPANLKGYFQVNQNSTSSHLGSS

SEQ ID NO: 8 - DNA, 1155 bp, Artificial Sequence
  TPST2
ggatccgccaccatgagactgtccgtgcgccgggtgctgctggccgctggatgcgcactggtgctggtgctggcagtgcagctgggacagcaggtgctggagtgtagggccgtgctggcaggcctgcggtcccctcgcggcgccatgcgcccagagcaggaggagctggtcatggtgggcaccaatcacgtggagtaccggtatggcaaggccatgccactgatcttcgtgggcggcgtgcctcggtctggaaccacactgatgagggcaatgctggacgcacacccagaggtgaggtgcggagaggagacaaggatcatcccccgcgtgctggcaatgagacaggcatggtccaagtctggcagggagaagctgagactggatgaggccggcgtgacagacgaggtgctggatgccgccatgcaggccttcatcctggaagtgatcgcaaagcacggagagccagcccgggtgctgtgcaataaggacccctttaccctgaagagctccgtgtacctgtcccgcctgttccccaactctaagtttctgctgatggtgagggatggcagagcctctgtgcacagcatgatcaccaggaaggtgacaatcgccggcttcgacctgtctagctacagagattgtctgacaaagtggaataaggccatcgaagtgatgtatgcccagtgcatggaagtgggcaaggagaagtgtctgcccgtgtactatgagcagctggtgctgcaccctaggagaagcctgaagctgatcctggactttctgggaatcgcatggtccgacgccgtgctgcaccacgaggatctgatcggcaagcccggcggcgtgagcctgtccaagatcgagaggtctaccgatcaggtcatcaagcctgtgaacctggaggccctgagcaagtggacaggccacatcccaggcgacgtggtgagagatatggcccagatcgcccctatgctggcccagctgggctacgacccatatgccaaccctcccaattacggcaacccagatccctttgtgatcaacaatacccagcgcgtgctgaagggcgactataagacacctgccaatctgaagggctatttccaggtcaaccagaactcaacatcctctcatctggggagcagttgataactcgag

SEQ ID NO: 9 - protein, 360 aa, Artificial Sequence
  TPST2 delta TM
MRLSVRRVQQVLECRAVLAGLRSPRGAMRPEQEELVMVGTNHVEYRYGKAMPLIFVGGVPRSGTTLMRAMLDAHPEVRCGEETRIIPRVLAMRQAWSKSGREKLRLDEAGVTDEVLDAAMQAFILEVIAKHGEPARVLCNKDPFTLKSSVYLSRLFPNSKFLLMVRDGRASVHSMITRKVTIAGFDLSSYRDCLTKWNKAIEVMYAQCMEVGKEKCLPVYYEQLVLHPRRSLKLILDFLGIAWSDAVLHHEDLIGKPGGVSLSKIERSTDQVIKPVNLEALSKWTGHIPGDVVRDMAQIAPMLAQLGYDPYANPPNYGNPDPFVINNTQRVLKGDYKTPANLKGYFQVNQNSTSSHLGSS

SEQ ID NO: 10 - DNA, 1104 bp, Artificial Sequence
  TPST2 delta TM
ggatccgccaccatgagactgtccgtgcgccgggtgcagcaggtgctggagtgtagggccgtgctggcaggcctgcggtcccctcgcggcgccatgcgcccagagcaggaggagctggtcatggtgggcaccaatcacgtggagtaccggtatggcaaggccatgccactgatcttcgtgggcggcgtgcctcggtctggaaccacactgatgagggcaatgctggacgcacacccagaggtgaggtgcggagaggagacaaggatcatcccccgcgtgctggcaatgagacaggcatggtccaagtctggcagggagaagctgagactggatgaggccggcgtgacagacgaggtgctggatgccgccatgcaggccttcatcctggaagtgatcgcaaagcacggagagccagcccgggtgctgtgcaataaggacccctttaccctgaagagctccgtgtacctgtcccgcctgttccccaactctaagtttctgctgatggtgagggatggcagagcctctgtgcacagcatgatcaccaggaaggtgacaatcgccggcttcgacctgtctagctacagagattgtctgacaaagtggaataaggccatcgaagtgatgtatgcccagtgcatggaagtgggcaaggagaagtgtctgcccgtgtactatgagcagctggtgctgcaccctaggagaagcctgaagctgatcctggactttctgggaatcgcatggtccgacgccgtgctgcaccacgaggatctgatcggcaagcccggcggcgtgagcctgtccaagatcgagaggtctaccgatcaggtcatcaagcctgtgaacctggaggccctgagcaagtggacaggccacatcccaggcgacgtggtgagagatatggcccagatcgcccctatgctggcccagctgggctacgacccatatgccaaccctcccaattacggcaacccagatccctttgtgatcaacaatacccagcgcgtgctgaagggcgactataagacacctgccaatctgaagggctatttccaggtcaaccagaactcaacatcctctcatctggggagcagttgataactcgag

SEQ ID NO: 11 - protein, 18 aa, Artificial Sequence
  IgE signal peptide
MDWTWILFLVAAATRVHS

SEQ ID NO: 12 - protein, 5 aa, Artificial Sequence
  dileucine motif
  misc_feature (2)..(2): any amino acid
  misc_feature (3)..(3): any amino acid
DXXLL

SEQ ID NO: 13 - protein, 4 aa, Artificial Sequence
  tyrosine-based motif
  misc_feature (2)..(2): any amino acid
  misc_feature (3)..(3): any amino acid
  misc_feature (4)..(4): an amino acid with a bulky hydrophobic side chain
YXXX

(19) United States
(12) Patent Application Publication
(10) Pub. No.: US 2021/0244795 A1
(43) Pub. Date: Aug. 12, 2021

(54) COMPOSITIONS AND METHODS FOR IN VIVO POST TRANSLATIONAL MODIFICATION

(71) Applicant: THE WISTAR INSTITUTE OF ANATOMY AND BIOLOGY, Philadelphia, PA (US)

(72) Inventors: David Weiner, Merion, PA (US); Megan Wise, Glenside, PA (US); Ziyang Xu, Philadelphia, PA (US)

(21) Appl. No.: 16/973,603

(22) PCT Filed: Jun. 11, 2019
(86) PCT No.: PCT/US19/36470

§ 371 Date: Dec. 9, 2020

Related U.S. Application Data
(60) Provisional application No. 62/683,344, filed on Jun. 11, 2018.

(51) Int. Cl. A61K 38/17; A61K 38/45; A61K 41/00

(52) U.S. Cl. CPC A61K 38/1774; A61K 41/0047; C12Y 208/0202; A61K 38/45

(57) ABSTRACT

Disclosed herein are compositions and methods for post-translationally modifying synthetic biologics in vivo.

### CROSS-REFERENCE TO RELATED APPLICATIONS

This application is entitled to priority to U.S. Provisional Application No. 62/683,344, filed Jun. 11, 2018, which is incorporated by reference herein in its entirety.

### BACKGROUND

The activity of many proteins can be improved with post-translational modifications. A post translational modification (PTM) is a chemical change that results in the covalent attachment of different functional groups to the protein. These modifications can specifically target said protein to certain cellular pathways, improved overall function and potency, change stability or half-life, or lead to differences in folding and other protein interactions. The ability to encode different target proteins has been well established using DNA plasmids. However, the need to further improve these DNA encoded proteins is necessary. By encoding different enzymes which can perform post-translational modifications, the target protein can be modified and thus improve the overall desired outcome. This additional step of regulation allows for the specific tailoring of encoded proteins and could be used for both vaccine as well as other DNA encoded proteins.

Currently, biologics (antibodies, erythropoietin, clotting factors) used as pharmaceuticals are frequently produced in mammalian cell lines (CHO) and display heterogeneity in terms of location and extent of PTMs, some of which might decrease the functionality of the biologics (Harris, 2005). It would therefore be a major advantage to have a simple approach to facilitate in vivo delivery and modifications of these complex biological molecules using advanced DNA/electroporation (EP) technology.

### SUMMARY

The present invention provides methods of post-translationally modifying a synthetic protein in a subject. In one embodiment, the method comprises administering to the subject a composition comprising a first recombinant nucleic acid sequence encoding the synthetic protein, and a second recombinant nucleic acid sequence encoding a modifier protein, wherein the modifier protein post-translationally modifies the synthetic biologic in the subject.

In one embodiment, the post translational modification is selected from the group consisting of sulfation, acetylation, N-linked glycosylation, myristoylation, palmitoylation, SUMOylation, hydroxylation, methylation, O-linked glycosylation, ubiquitylation, oxidation, and palmitoylation.

In one embodiment, the post translational modification is sulfation and the modifier protein is selected from the group consisting of tyrosylprotein sulfotransferase 1 (TPST1) and TPST2.

In one embodiment, the modifier protein is TPST2. In one embodiment, TPST2 comprises an IgE leader. In one embodiment, TPST2 comprises an amino acid sequence at least 90% homologous to SEQ ID NO: 5 or 7. In one embodiment, the second recombinant nucleic acid sequence comprises a sequence at least 90% homologous to SEQ ID NO: 6 or 8.

In one embodiment, the synthetic protein is an antigen, antibody or immunoadhesin. In one embodiment, the immunoadhesin is eCD4-Ig. In one embodiment, eCD4-Ig comprises an amino acid sequence at least 90% homologous to SEQ ID NO:1 or 3. In one embodiment, the first recombinant nucleic acid sequence comprises a sequence at least 90% homologous to SEQ ID NO:2 or 4.

In one embodiment, the post translational modification is sulfation, the modifier protein is tyrosylprotein sulfotransferase 1 (TPST2), and the synthetic protein is eCD4-Ig, wherein eCD4-Ig is sulfated in the subject.

The invention also provides a composition for post-translationally modifying a synthetic protein in a subject. In one embodiment, the composition comprises a first recombinant nucleic acid sequence encoding the synthetic protein and a second recombinant nucleic acid sequence encoding a modifier protein.

In one embodiment, the modifier protein catalyzes a post translational modification (PTM) on the synthetic protein, wherein the PTM is selected from the group consisting of post translational modification is selected from the group consisting of sulfation, acetylation, N-linked glycosylation, myristoylation, palmitoylation, SUMOylation, hydroxylation, methylation, O-linked glycosylation, ubiquitylation, oxidation, and palmitoylation

In one embodiment, the post translational modification is sulfation and the modifier protein is selected from the group consisting of tyrosylprotein sulfotransferase 1 (TPST1) and TPST2.

In one embodiment, the modifier protein is TPST2. In one embodiment, TPST2 comprises an IgE leader. In one embodiment, TPST2 comprises an amino acid sequence at least 90% homologous to SEQ ID NO: 5 or 7. In one embodiment, the second recombinant nucleic acid sequence comprises a sequence at least 90% homologous to SEQ ID NO: 6 or 8.

In one embodiment, the synthetic protein is an antigen, antibody or immunoadhesin. In one embodiment, the immunoadhesin is eCD4-Ig. In one embodiment, eCD4-Ig comprises an amino acid sequence at least 90% homologous to SEQ ID NO:1 or 3. In one embodiment, the first recombinant nucleic acid sequence comprises a sequence at least 90% homologous to SEQ ID NO:2 or 4.

In one embodiment, the one or more nucleic acid molecules are engineered to be in an expression vector. In one embodiment, the composition comprises a pharmaceutically acceptable excipient.

In one embodiment, the invention provides a method for treating a disease, disorder or infection in a subject in need thereof. In one embodiment, the method comprises administering a composition of the invention to the subject. In one embodiment, the method comprises an electroporation step.

### BRIEF DESCRIPTION OF THE DRAWINGS

FIG. 1, comprising FIG. 1A through FIG. 1F, depicts in vitro expression and sulfation of ReCD4-Ig. FIG. 1A depicts the expression of ReCD4-Ig in transfection lysate of HEK293T cells normalized to total protein concentrations. FIG. 1B depicts the expression of ReCD4-Ig in transfection supernatant of HEK293T cells. FIG. 1C depicts a western blot of supernatants of HEK293T cells transfected with either p-ReCD4-Ig or pVAX (plasmid backbone vector). FIG. 1D depicts a binding ELISA to detect tyrosine sulfation of ReCD4-Ig in transfection supernatant. FIG. 1E depicts quantification of ReCD4-Ig in supernatants of HEK293T cells transfected with p-ReCD4-Ig and varying doses of p-IgE-TPST2. Pairwise T-tests were used to compare level between each group with no enzyme group, and significant reduction (p<0.05) was detected in the 1:20 IgE-TPST2 group. FIG. 1F depicts a western blot of supernatants of HEK293T cells transfected with either p-ReCD4-Ig alone or p-ReCD4-Ig and 1:1000 plasmid enzymes. Lower panel is loading control to demonstrate the same amount of ReCD4-Ig in transfection supernatant.

FIG. 2, comprising FIG. 2A through FIG. 2C, depicts experimental results demonstrating the subcellular targeting of IgE-TPST2. FIG. 2A depicts confocal microscopy to determine localization between TPST2 variant (red) and Golgin 97 (green). Nuclei are stained with DAPI (blue). IgE-TPST2 demonstrates increased trafficking to TGN as compared to TPST2. Diffuse cytoplasmic distribution of ATM-TPST2 can be observed in the last panel. FIG. 2B depicts localization between TPST2 variant and Golgin 97 was quantified with regions of interest analyses (n=16). One-way ANOVA (F-statistic=79.67, p-value <0.0001) and post-hoc pairwise T-tests (with Holm's adjustment) were used to compare Pearson's correlation coefficients between different groups. P-values are as indicated. FIG. 2C depicts fluorescence microscopy images of HEK293T cells transfected with pGX00001 backbone plasmid alone, or pGX00001 with plasmid encoded enzymes. Overlay of the three channels show enhanced trafficking of IgE-TPST2 to TGN as compared to TPST2 and ATM-TPST2.

FIG. 3, comprising FIG. 3A through FIG. 3F, depicts In vivo expression and sulfation of ReCD4-Ig. FIG. 3A depicts a western blot of muscle homogenates 7 d.p.i demonstrates expression of IgE-TPST2 (43 kDa) in the injected legs as compared to the contralateral legs. GAPDH (37 kDa) serves as loading controls. FIG. 3B depicts serum expression of ReCD4-Ig in B6.Cg-Foxn1nu/J and balb/c transiently immuno-modulated injected with a single dose of DNA. FIG. 3C depicts a binding ELISA to determine ReCD4-Ig tyrosine sulfation in mice sera. Transiently depleted balb/c mice were injected with p-ReCD4-Ig and varying doses of p-IgE-TPST2; sera 7 d.p.i were collected for analyses. OD450 of each group was compared to the no-enzyme and 1:20IgE-TPST2 groups to determine minimal dose of IgE-TPST2 required for sulfation. FIG. 3D depicts serum expression level of ReCD4-Ig in mice co-treated with p-ReCD4-Ig and varying plasmid enzyme doses 7 d.p.i. P-values were computed with pairwise T-tests: * p<0.05, ** p<0.005, *** p<0.0005, ***** p<0.00005, ***** p<0.000005. FIG. 3E depicts serum expression level of ReCD4-Ig at different time points in transiently depleted balb/c treated with either p-ReCD4-Ig or p-ReCD4-Ig and 1:1000 dose of p-IgE-TPST2. Each line represents an individual mouse. FIG. 3F depicts serum concentrations of ReCD4-Ig 7 d.p.i in transiently depleted balb/c mice injected with p-ReCD4-Ig alone or p-ReCD4-Ig+p-TPST1/p-HS3SA. Significant reductions (p<0.05, pairwise T-tests with Holm adjustment) in expression levels were observed in both groups when compared to ReCD4-Ig only group. *, p<0.05, **, p<0.005.

FIG. 4, comprising FIG. 4A through FIG. 4F, depicts the functional characterization of IgE-TPST2 mediated sulfation of ReCD4-Ig. FIG. 4A depicts serum concentrations of ReCD4-Ig at the time of terminal bleed (7 d.p.i) in transiently depleted balb/c mice injected with p-ReCD4-Ig alone or p-ReCD4-Ig+p-IgE-TPST2. FIG. 4B depicts experimental results demonstrating the neutralization of 25710 pseudotyped virus versus serum concentration of ReCD4-Ig. Error bar represents standard deviation. FIG. 4C depicts a comparison of ReCD4-Ig IC50 with or without sulfation for HIV pseudotyped viruses. FIG. 4D depicts IC50 values (mean±standard deviation) of ReCD4-Ig in sera of mice with and without IgE-TPST2 treatment. Geometric mean of IC50 across the panel (except for MLV) is also given for comparison. FIGS. 4E and 4F depicts IC50 values of ReCD4-Ig with or without sulfation in ex vivo neutralization assay. Each dot represents IC50 value computed from a single mouse, and p-value for each virus is computed with pairwise T-test with Holm adjustment for multiple comparisons. P-value of less than 0.05 is considered significant. FIG. 4E depicts IC50 values of viruses in which sulfated ReCD4-Ig has significantly enhanced potency. FIG. 4F depicts IC50 values of in which the potency of sulfated ReCD4-Ig is not significantly higher.

FIG. 5, comprising FIG. 5A through FIG. 5D, depicts experimental results of expression of immunoadhesin in an alternative model of NSG SCID mice that had been reconstituted with human immune system through transplantation of human fetal thymus implants and administration with a series of DNA-encoded cytokines. FIG. 5A depicts in vivo expression of ReCD4-Ig in these humanized mice that had received 320 ug of DNA-encoded ReCD4-Ig along with 0.32 ug of p-IgE-TPST2, as demonstrated by ELISA binding to JR-FL GP120 using sera from various timepoints. FIG. 5B depicts sera concentration of ReCD4-Ig in each individual mouse over the course of 35 days post injection (d.p.i); peak expression is observed at 14 d.p.i; each line represents an individual mouse. FIG. 5C depicts ex vivo neutralization of Tier 1 SF162 pseudovirus by mice sera prior to (in blue) and post (in red) DNA treatment. FIG. 5D. depicts neutralization of Tier 1 virus SF162, and Tier 2 viruses THRO and JR-FL by D7 sera of the mice in terms of IC50 neutralization titers; each mouse represents an individual mouse; mean and standard deviations in the titers are also shown.

### DETAILED DESCRIPTION

The invention is partly based on the use of nucleic acid sequences to encode an enzyme for post-translational modification (PTM) of a target protein for production directly in vivo. In one embodiment, the present invention relates to compositions and methods for post-translationally modifying a synthetic protein in a subject. In one aspect, the invention provides a composition comprising a first recombinant nucleic acid sequence encoding the synthetic protein, and a second recombinant nucleic acid sequence encoding a modifier protein

The composition can be administered to a subject in need thereof to facilitate in vivo expression, formation, and post-translationally modification of a synthetic protein.

In particular, the synthetic protein and modifier protein from the recombinant nucleic acid sequences are expressed and the modifier protein post-translationally modifies the synthetic protein. The post-translationally modified synthetic protein has increased biologic activity as compared to a protein not expressed and modified as described herein.

In one embodiment, the wherein the modifier protein catalyzes a post translational modification (PTM) on the synthetic protein, wherein the PTM is selected from the group consisting of post translational modification is selected from the group consisting of sulfation, acetylation, N-linked glycosylation including sialylation and fucosylation/defucosylation, myristoylation, palmitoylation, SUMOylation, hydroxylation, methylation, O-linked glycosylation, ubiquitylation, oxidation, amidation and palmitoylation. Examples of PTM might include but not limited to Table 1.

TABLE 1
Examples of DNA-encoded enzymes that can carry out PTM of a target protein for improved
functions

| | Post-translational | | |
|---|---|---|---|
| Protein target | Modification (PTM) | Function | Enzymes that mediate PTM |
| Erythropoietin | Terminal sialyation | Improves protein | Uridine diphosphate-N-acetyl |
| | | half-life and in vivo | glucosamine 2-epimerase)/MNK |
| | biological activity | (N-acetyl mannosamine | |
| | | kinase (Delorme et al., | with R263L-R266Q mutation |
| | | 1992) | (Son et al., 2011) |
| IgG1 class | N-linked bisected | Improves ADCC | beta (1,4)-N- |
| immunoglobulins | oligosaccharides | | acetylglucosaminyltransferase III |
| | | | (Umana et al., 1999) |
| Clotting Factor VII, IX | Glutamate y- | Facilitate binding | y-glutamyl carboxylase (GGCX) |
| and X; Protein C | Carboxylation | to Ca2+ and | and |
| | | biological | vitamin K oxidoreductase (VKOR) |
| | | functions | (Sun et al., 2005) |
| Hirudin (Anti- | Tyr-63 Sulfation | Improves affinity | TPST1/TPST2 |
| coagulant) | | for thrombin by | (Walsh and Jefferis, 2006) |
| | | 10-fold | |
| | | (Stone and | |
| | | Hofsteenge, 1986) | |
| Factor VIII | Tyrosine sulfation | Facilitates | TPST1/TPST2 |
| | | conversion from | (Walsh and Jefferis, 2006) |
| | | Factor VIII to Villa | |
| | | and binding to | |
| | | carrier von | |
| | | Willibrand factor | |
| | | (Tsang et al., 1988) | |
| Calcitonin | C-terminal | Enhances ligand- | Peptidylglycine |
| | amidation | receptor | alpha-amidating |
| | | interaction | monooxygenase (PAM) |
| | | (Bradbury and | (Prigge et al., 2000) |
| | | Smyth, 1991) | |

In one aspect, the present invention relates to a composition that can be used to treat a disease or disorder by administering the engineered synthetic proteins (e.g., synthetic protein and modifier protein in the form of synthetic nucleic acid plasmids).

In one aspect, the present invention relates to compositions comprising a first recombinant nucleic acid sequence encoding a synthetic eCD4-Ig, and a second recombinant nucleic acid sequence encoding a synthetic tyrosylprotein sulfotransferase (TPST). In one embodiment, the TPST is TPST1 or TPST2. In one embodiment, the TPST is TPST2. In one embodiment TPST comprises an IgE leader. The composition can be administered to a subject in need thereof to facilitate in vivo expression, formation and sulfation of eCD4-Ig.

In one embodiment, the first recombinant nucleic acid sequence encoding a synthetic eCD4-Ig encodes a sequence at least 90% homologous to SEQ ID NO:1 or 3, or fragment thereof. In one embodiment, the first recombinant nucleic acid sequence comprises a sequence at least 90% homologous to SEQ ID NO:2 or 4, or fragment thereof.

In one embodiment, the second recombinant nucleic acid sequence encoding a TPST2 encodes a sequence at least 90% homologous to SEQ ID NO:5 or 7, or fragment thereof. In one embodiment, the second recombinant nucleic acid sequence comprises a sequence at least 90% homologous to SEQ ID NO:6 or 8, or fragment thereof.

In one embodiment, the second recombinant nucleic acid sequence comprises sequence encoding the polypeptide sequence at least 90% homologous to SEQ ID NOs: 5 or 7, or a fragment thereof. In one embodiment, the second recombinant nucleic acid sequence comprises an RNA sequence transcribed from a DNA sequence described herein. For example, in one embodiment, the second recombinant nucleic acid sequence comprises an RNA sequence transcribed by a DNA sequence encoding the polypeptide sequence at least 90% homologous to SEQ ID NOs: 5 or 7, or a fragment thereof.

In one embodiment, the second recombinant nucleic acid sequence encodes an amino acid sequence having at least 90% homology to SEQ ID NO: 5 or 7. In one embodiment, the second recombinant nucleic acid sequence encodes a fragment of an amino acid sequence having at least 90% homology to SEQ ID NO: 5 or 7. In one embodiment, the second recombinant nucleic acid sequence comprises a sequence at least 90% homologous to SEQ ID NO:6 or 8, or a fragment thereof.

The compositions provided herein can also include a pharmaceutically acceptable excipient.

Aspects of the invention also include methods for treating a disease, disorder or infection in a subject in need thereof by administering any of the compositions provided herein to the subject. In one embodiment, the infection is an HIV infection. The methods of increasing an immune response can also include an electroporating step.

### 1. DEFINITIONS

Unless otherwise defined, all technical and scientific terms used herein have the same meaning as commonly understood by one of ordinary skill in the art. In case of conflict, the present document, including definitions, will control. Exemplary methods and materials are described herein, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Antibody” may mean an antibody of classes IgG, IgM, IgA, IgD or IgE, or fragments, fragments or derivatives thereof, including Fab, F(ab′)2, Fd, and single chain antibodies, and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

“Antigen” refers to proteins that have the ability to generate an immune response in a host. An antigen may be recognized and bound by an antibody. An antigen may originate from within the body or from the external environment.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). The structure and protein folding of the antibody may mean that other residues are considered part of the antigen binding region and would be understood to be so by a skilled person. See for example Chothia et al., (1989) Conformations of immunoglobulin hypervariable regions; Nature 342, p 877-883.

“Antibody fragment” or “fragment of an antibody” as used interchangeably herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Adjuvant” as used herein means any molecule added to the vaccine described herein to enhance the immunogenicity of the antigen.

“Coding sequence” or “encoding nucleic acid” as used herein may refer to the nucleic acid (RNA or DNA molecule) that comprise a nucleotide sequence which encodes an antibody as set forth herein. The coding sequence may also comprise a DNA sequence which encodes an RNA sequence. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides.

“Complement” or “complementary” as used herein may mean a nucleic acid may have Watson-Crick (e.g., A-T/U and C-G) or Hoogsteen base pairing between nucleotides or nucleotide analogs of nucleic acid molecules.

“Consensus” or “consensus sequence” as used herein means a polypeptide sequence based on analysis of an alignment of multiple sequences of multiple subtypes of a particular antigen. Nucleic acid sequences that encode a consensus polypeptide sequence can be prepared. Vaccines or immunological compositions comprising proteins that comprise consensus sequences and/or nucleic acid molecules that encode such proteins can be used to induce broad immunity against multiple subtypes or serotypes of a particular antigen.

“Constant current” as used herein to define a current that is received or experienced by a tissue, or cells defining said tissue, over the duration of an electrical pulse delivered to same tissue. The electrical pulse is delivered from the electroporation devices described herein. This current remains at a constant amperage in said tissue over the life of an electrical pulse because the electroporation device provided herein has a feedback element, preferably having instantaneous feedback. The feedback element can measure the resistance of the tissue (or cells) throughout the duration of the pulse and cause the electroporation device to alter its electrical energy output (e.g., increase voltage) so current in same tissue remains constant throughout the electrical pulse (on the order of microseconds), and from pulse to pulse. In some embodiments, the feedback element comprises a controller.

“Current feedback” or “feedback” as used herein may be used interchangeably and may mean the active response of the provided electroporation devices, which comprises measuring the current in tissue between electrodes and altering the energy output delivered by the EP device accordingly in order to maintain the current at a constant level. This constant level is preset by a user prior to initiation of a pulse sequence or electrical treatment. The feedback may be accomplished by the electroporation component, e.g., controller, of the electroporation device, as the electrical circuit therein is able to continuously monitor the current in tissue between electrodes and compare that monitored current (or current within tissue) to a preset current and continuously make energy-output adjustments to maintain the monitored current at preset levels. The feedback loop may be instantaneous as it is an analog closed-loop feedback.

“Decentralized current” as used herein may mean the pattern of electrical currents delivered from the various needle electrode arrays of the electroporation devices described herein, wherein the patterns minimize, or preferably eliminate, the occurrence of electroporation related heat stress on any area of tissue being electroporated.

“Electroporation,” “electro-permeabilization,” or “electro-kinetic enhancement” (“EP”) as used interchangeably herein may refer to the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Endogenous antibody” as used herein may refer to an antibody that is generated in a subject that is administered an effective dose of an antigen for induction of a humoral immune response.

“Feedback mechanism” as used herein may refer to a process performed by either software or hardware (or firmware), which process receives and compares the impedance of the desired tissue (before, during, and/or after the delivery of pulse of energy) with a present value, preferably current, and adjusts the pulse of energy delivered to achieve the preset value. A feedback mechanism may be performed by an analog closed loop circuit.

“Fragment” or “immunogenic fragment” as used herein, a nucleic acid sequence or amino acid sequence. In one embodiment, the fragment is a nucleic acid sequence that encodes a fragment of a protein, such as an antibody or antigen. The fragment may be a fragment of a protein, antibody or antigen, which retains its biologic activity. “Fragment” or “immunogenic fragment” may also mean a fragment of a nucleic acid molecule. The fragments can be DNA fragments of the various nucleotide sequences that encode protein fragments. The fragments can be DNA fragments of DNA sequences having homology to at least one of the various nucleotide sequences that encode protein fragments set forth below. A fragment of an protein or nucleic acid may be 100% identical to the full length except missing at least one amino acid/nucleic acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full-length protein or nucleic acid, excluding any heterologous signal peptide added. The fragment may comprise a fragment of a polypeptide that is 95% or more, 96% or more, 97% or more, 98% or more or 99% or more identical to the protein or nucleic acid and additionally comprise an N terminal methionine or heterologous signal peptide which is not included when calculating percent identity. Fragments may further comprise an N terminal methionine and/or a signal peptide such as an immunoglobulin signal peptide, for example an IgE or IgG signal peptide. The N terminal methionine and/or signal peptide may be linked to a fragment of an antibody.

A fragment of a nucleic acid sequence may be 100% identical to the full length except missing at least one nucleotide from the 5′ and/or 3′ end. When the nucleic acid sequence encodes a protein, the fragment of the nucleic acid sequence may be, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antibody and additionally optionally comprise sequence encoding an N terminal methionine or heterologous signal peptide which is not included when calculating percent identity. Fragments may further comprise coding sequences for an N terminal methionine and/or a signal peptide such as an immunoglobulin signal peptide, for example an IgE or IgG signal peptide. The coding sequence encoding the N terminal methionine and/or signal peptide may be linked to a fragment of coding sequence.

“Genetic construct” as used herein refers to the DNA or RNA molecules that comprise a nucleotide sequence which encodes a protein, such as an antibody. The genetic construct may also refer to a DNA molecule which transcribes an RNA. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

The term “homology,” as used herein, refers to a degree of complementarity. There can be partial homology or complete homology (i.e., identity). A partially complementary sequence that at least partially inhibits a completely complementary sequence from hybridizing to a target nucleic acid is referred to using the functional term “substantially homologous.” When used in reference to a double-stranded nucleic acid sequence such as a cDNA or genomic clone, the term “substantially homologous,” as used herein, refers to a probe that can hybridize to a strand of the double-stranded nucleic acid sequence under conditions of low stringency. When used in reference to a single-stranded nucleic acid sequence, the term “substantially homologous,” as used herein, refers to a probe that can hybridize to (i.e., is the complement of) the single-stranded nucleic acid template sequence under conditions of low stringency.

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Impedance” as used herein may be used when discussing the feedback mechanism and can be converted to a current value according to Ohm's law, thus enabling comparisons with the preset current.

“Immune response” as used herein may mean the activation of a host's immune system, e.g., that of a mammal, in response to the introduction of one or more nucleic acids and/or peptides. The immune response can be in the form of a cellular or humoral response, or both.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV 40 late promoter and the CMV IE promoter.

“Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein may facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence dependent and will be different in different circumstances. Stringent conditions may be selected to be about 5-10° C. lower than the thermal melting point (Tm) for the specific sequence at a defined ionic strength pH. The Tm may be the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc) and a human). In some embodiments, the subject may be a human or a non-human. The subject or patient may be undergoing other forms of treatment.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

“Synthetic antibody” as used herein refers to an antibody that is encoded by the recombinant nucleic acid sequence described herein and is generated in a subject.

“Synthetic biologic” as used herein refers to a protein that is encoded by the recombinant nucleic acid sequence described herein and is generated in a subject or a recombinant nucleic acid sequence that is administered to a subject.

“Treatment” or “treating,” as used herein can mean protecting of a subject from a disease through means of preventing, suppressing, repressing, or completely eliminating the disease. Preventing the disease involves administering a vaccine of the present invention to a subject prior to onset of the disease. Suppressing the disease involves administering a vaccine of the present invention to a subject after induction of the disease but before its clinical appearance. Repressing the disease involves administering a vaccine of the present invention to a subject after clinical appearance of the disease.

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or sequences substantially identical thereto.

“Variant” with respect to a peptide or polypeptide, may indicate that the peptide or polypeptide differs in amino acid sequence by the insertion, deletion, or conservative substitution of amino acids, but retains at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. This applies regardless of the breadth of the range.

### 2. COMPOSITIONS

In one aspect, the invention provides compositions for generating a biologic in a subject and post-translationally modifying the biologic in the subject. In one embodiment, the composition comprises a first nucleic acid sequence and a second nucleic acid sequence.

In one embodiment, the first nucleic acid encodes a biologic. In one embodiment, the first nucleic acid sequence encodes a protein. In one embodiment, the first nucleic acid sequence encodes a synthetic antigen, a synthetic antibody, or a synthetic protein. In one embodiment, the first nucleic acid sequence encodes an immunoadhesin.

In one embodiment, the second nucleic acid encodes a modifying protein. In one embodiment, the modifying protein post-translationally modifies the protein encoded by the first nucleic acid sequence. In one embodiment, the modifying protein modifies the first nucleic acid sequence.

In one aspect, the present invention relates to compositions comprising a first recombinant nucleic acid sequence encoding a synthetic eCD4-Ig, and a second recombinant nucleic acid sequence encoding a synthetic tyrosylprotein sulfotransferase (TPST). In one embodiment, the TPST is TPST1 or TPST2. In one embodiment, the TPST is TPST2. In one embodiment TPST comprises an IgE leader. The composition can be administered to a subject in need thereof to facilitate in vivo expression, formation and sulfation of eCD4-Ig.

In one embodiment, the first recombinant nucleic acid sequence encoding a synthetic eCD4-Ig encodes a sequence at least 90% homologous to SEQ ID NO:1 or 3, or fragment thereof. In one embodiment, the first recombinant nucleic acid sequence comprises a sequence at least 90% homologous to SEQ ID NO:2 or 4, or fragment thereof.

In one embodiment, the first recombinant nucleic acid sequence comprises sequence encoding the polypeptide sequence at least 90% homologous to SEQ ID NOs: 1 or 3, or a fragment thereof. In one embodiment, the first recombinant nucleic acid sequence comprises an RNA sequence transcribed from a DNA sequence described herein. For example, in one embodiment, the first recombinant nucleic acid sequence comprises an RNA sequence transcribed by a DNA sequence encoding the polypeptide sequence at least 90% homologous to SEQ ID NOs: 1 or 3, or a fragment thereof.

In one embodiment, the second recombinant nucleic acid sequence encodes an amino acid sequence having at least 90% homology to SEQ ID NO: 1 or 3. In one embodiment, the second recombinant nucleic acid sequence encodes a fragment of an amino acid sequence having at least 90% homology to SEQ ID NO: 1 or 3. In one embodiment, the second recombinant nucleic acid sequence comprises a sequence at least 90% homologues to SEQ ID NO:2 or 4.

In one embodiment, the second recombinant nucleic acid sequence encoding a TPST2 encodes a sequence at least 90% homologous to SEQ ID NO:5 or 7, or fragment thereof. In one embodiment, the second recombinant nucleic acid sequence comprises a sequence at least 90% homologous to SEQ ID NO:6 or 8, or fragment thereof.

In one embodiment, the second recombinant nucleic acid sequence comprises sequence encoding the polypeptide sequence at least 90% homologous to SEQ ID NOs: 5 or 7, or a fragment thereof. In one embodiment, the second recombinant nucleic acid sequence comprises an RNA sequence transcribed from a DNA sequence described herein. For example, in one embodiment, the second recombinant nucleic acid sequence comprises an RNA sequence transcribed by a DNA sequence encoding the polypeptide sequence at least 90% homologous to SEQ ID NOs: 5 or 7, or a fragment thereof.

In one embodiment, the second recombinant nucleic acid sequence encodes an amino acid sequence having at least 90% homology to SEQ ID NO: 5 or 7. In one embodiment, the second recombinant nucleic acid sequence encodes a fragment of an amino acid sequence having at least 90% homology to SEQ ID NO: 5 or 7. In one embodiment, the second recombinant nucleic acid sequence comprises a sequence at least 90% homologues to SEQ ID NO:6 or 8.

The compositions provided herein can also include a pharmaceutically acceptable excipient.

### 3. MODIFIER PROTEIN

Provided herein are proteins capable of post-translationally modifying a protein or nucleic acid in a subject. For example, in one embodiment, the modifier proteins described herein can be used to post-translationally modify a protein which is required for biologic activity. In one embodiment, the modifier proteins described herein can post-translationally modifies a protein, wherein the modification includes, but is not limited to, sulfation, acetylation, N-linked glycosylation including sialylation and fucosylation/defucosylation, myristoylation, palmitoylation, SUMOylation, amidation, hydroxylation, methylation, O-linked glycosylation, ubiquitylation, pyrrolidone Carboxylic Acid, deamination, isomerization, oxidation, palmitoylation, and cyclization (Table 1).

Exemplary sulfation enzymes include tyrosylprotein sulfotransferase (TPST). Exemplary acetylation enzymes include, but are not limited to, NatA, NatB, NatC, NatD, NatE, NatF. acetyl-coenzyme A, histone acetyltransferase, and histone deacetylase. Exemplary deamidation enzymes include, but are not limited to, O-acyltransferase. Exemplary myristoylation enzymes include, but are not limited to, N-myristoyltransferase (NMT). Exemplary ubiquitylation enzymes include, but are not limited to, ubiquitin-activating enzymes, ubiquitin-conjugating enzymes, and ubiquitin ligases. Exemplary SUMOylation enzymes include, but are not limited to, SUMO-1, SUMO-2, SUMO-3 and SUMO-4. Exemplary methylation enzymes include, but are not limited to, Catechol-O-methyl transferase, DNA methyltransferase, Histone methyltransferase, 5-Methyltetrahydrofolate-homocysteine methyltransferase, O-methyltransferase, methionine synthase, and corrinoid-iron sulfur protein. Exemplary hydroxylation enzymes include, but are not limited to, prolyl 4-hydroxylase, prolyl 3-hydroxylase and lysyl 5-hydroxylase. Phsphorylation enzymes include kinases such as MAP kinases, AGC kinases, CaM kinases, CK1, CDK, GSK3 CLK, STE, Tyroxine kinases, and TKL. Exemplary N-glycosylation exymes include but are not limited to Fut8, GMDS, GNT-III B4Galt1, SLC35A2, ST6Gal1, and MGAT3.

In one embodiment, the modifier protein may be modified to traffic to secretory compartment of cells. In one embodiment, the modifier protein may be modified to localize with the target biologic protein or nucleic acid to be modified in vivo. In some embodiments, the modifier protein may comprise a signal peptide from a different protein such as an immunoglobulin protein, for example an IgE signal peptide or an IgG signal peptide. In one embodiment, the IgE signal peptide comprises the sequence MDWTWILFLVAAATRVHS (SEQ ID NO:11)

In one embodiment, the modifier protein may be modified to traffic to the mitochondria. In one embodiment, the modifier protein may comprise an N-terminal peptide comprising 10-70 amino acids that form amphipathic helices. In one embodiment, the modifier protein may comprise an N-terminal peptide dileucine motif (DXXLL (SEQ ID NO:12)). In one embodiment, the modifier protein may comprise an N-terminal peptide tyrosine-based motif (YXXØ (SEQ ID NO:13)).

In one embodiment, the modifier protein may be modified to traffic to the lysosome. In one embodiment, the modifier protein may comprise the cytoplasmic tail of a transmembrane protein. In one embodiment, the modifier protein may comprise the cytoplasmic tail of a transmembrane protein on the N-terminus. In one embodiment, the modifier protein may comprise the cytoplasmic tail of a transmembrane protein on the C-terminus.

In one embodiment, the modifier protein may be modified to traffic to the nucleus. In one embodiment, the modifier protein may comprise a 5 basic positively charged amino acids. In one embodiment, the modifier protein may comprise a 5 basic positively charged amino acids on the N-terminus. In one embodiment, the modifier protein may comprise a 5 basic positively charged amino acids on the C-terminus.

In one embodiment, the modifier protein is a sulfation enzyme. In one embodiment, the sulfation enzyme is a tyrosylprotein sulfotransferase (TPST). In one embodiment, the sulfation enzyme is TPST1 or TPST2. In one embodiment, the sulfation enzyme is TPST2.

In one embodiment, TPST2 is modified to traffic to secretory compartment of cells. In one embodiment, TPST2 is modified to traffic to secretory compartment of cells to localize with the target biologic protein or nucleic acid to be modified in vivo. In one embodiment, TPST2 is modified to comprise an N-terminal IgE peptide.

In one embodiment, TPST2 comprises an amino acid sequence at least 90% homologous to SEQ ID NO: 5 or 7. In one embodiment, TPST2 comprises the amino acid sequence set forth in SEQ ID NO: 5 or 7. In one embodiment, TPST2 comprises the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO: 5 or 7.

Fragments of the TPST2 can comprise at least 10%, at least 20%, at least 30%, at least 40%, at least 50%, at least 60%, at least 70%, at least 80%, at least 90%, or at least 95% of one or more of the amino sequences of TPST2. In one embodiment, TPST2 comprises a fragment of TPST2. In one embodiment, the fragment of TPST2 can comprise a fragment of SEQ ID NO: 5 or 7. In one embodiment, the fragment of TPST2 can comprise a fragment of a protein having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO: 5 or 7.

Also provided herein are nucleic acid molecules comprising a nucleic acid sequence encoding a modifier protein described herein. Coding sequences encoding the proteins set forth herein may be generated using routine methods. Also described herein are isolated nucleic acids comprising nucleic acid sequences that encode proteins.

In one embodiment, coding sequences of the modifier proteins are provided. The modifier protein may have at least one post-translation modification activity that may modify a target biologic protein or nucleic acid. The nucleic acid sequences may optionally comprise coding sequences that encode a signal peptide such as for example an IgE or IgG signal peptide.

The nucleic acid sequence may encode a full-length protein. For example, the nucleic acid sequence may encode a full-length sulfation enzyme. In one embodiment, the nucleic acid sequence may comprise a sequence that encodes TPST2. In one embodiment, the nucleic acid sequence may comprise a sequence that encodes SEQ ID NO: 5 or 7, a variant thereof, or a fragment thereof. In one embodiment, the nucleic acid sequence may comprise a sequence at least 90% homologous to SEQ ID NO: 6 or 8. In one embodiment, the nucleic acid sequence may comprise SEQ ID NO: 6 or 8. In one embodiment, the nucleic acid sequence comprises an RNA sequence encoding a full-length protein. For example, nucleic acids may comprise an RNA sequence encoding a sulfation enzyme. In one embodiment, the nucleic acid sequence may comprise an RNA sequence that encodes TPST2. In one embodiment, nucleic acids may comprise an RNA sequence encoding more of SEQ ID NOs: 5 or 7, a variant thereof, a fragment thereof or any combination thereof.

The nucleic acid sequence may encode a fragment of a protein. Fragments of a full-length proteins can comprise at least 10%, at least 20%, at least 30%, at least 40%, at least 50%, at least 60%, at least 70%, at least 80%, at least 90%, or at least 95% of one or more of the full-length protein. For example, fragments of a nucleic acid encoding eCD4-Ig can comprise at least 10%, at least 20%, at least 30%, at least 40%, at least 50%, at least 60%, at least 70%, at least 80%, at least 90%, or at least 95% of one or more of the nucleic acid sequences set forth herein.

The nucleic acid sequence may encode a protein homologous to a biologic protein. For example, the nucleic acid sequence may encode a protein homologous to eCD4-Ig. Nucleic acid sequence may comprise a sequence that encodes a protein homologous to SEQ ID NOs: 5 or 7. In one embodiment, the sequence may comprise a sequence that encodes a protein having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:5 or 7. In one embodiment, the sequence may comprise a sequence that having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleotide sequence set forth in SEQ ID NO:6 or 8.

### 4. BIOLOGICS

Provided herein are biologics and nucleic acids encoding biologics. In one embodiment, the biologic is a protein or nucleic acid. In one embodiment, the biologic is a nucleic acid. In one embodiment, the biologic is a protein. In one embodiment, the biologic is a nucleic acid comprising a nucleic acid sequence encoding a protein. For example, in one embodiment, the first nucleic acid sequence encodes a synthetic antigen, a synthetic antibody, or a synthetic protein.

In one embodiment, the first nucleic acid sequence encodes an immunoadhesin. For example, in one embodiment, the immunoadhesin is eCD4-Ig. In one embodiment, eCD4-Ig comprises an amino acid sequence at least 90% homologous to SEQ ID NO:1 or 3. In one embodiment, eCD4-Ig comprises the amino acid sequence set forth in SEQ ID NO: 1 or 3. In one embodiment, eCD4-Ig comprises the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO: 1 or 3. In one embodiment, the first nucleic acid sequence comprises a sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleotide sequence set forth in SEQ ID NO: 2 or 4.

Fragments of the immunoadhesin can comprise at least 10%, at least 20%, at least 30%, at least 40%, at least 50%, at least 60%, at least 70%, at least 80%, at least 90%, or at least 95% of one or more of the amino sequences of eCD4-Ig. In one embodiment, eCD4-Ig comprises a fragment of eCD4-Ig. In one embodiment, the fragment of eCD4-Ig can comprise a fragment of SEQ ID NO:3. In one embodiment, the fragment of eCD4-Ig can comprise a fragment of a protein having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:1 or 3.

a. Proteins

Provided herein are biologic proteins capable of carrying out a biologic function. The proteins can treat, prevent, and/or protect against disease or infection, in the subject administered a composition of the invention. In one embodiment, the biologic protein is an antigen capable of eliciting an immune response in a mammal.

In some embodiments, the biologic proteins may comprise a signal peptide from a different protein such as an immunoglobulin protein, for example an IgE signal peptide or an IgG signal peptide.

Fragments of a full-length biologic proteins can comprise at least 10%, at least 20%, at least 30%, at least 40%, at least 50%, at least 60%, at least 70%, at least 80%, at least 90%, or at least 95% of one or more of the full-length sequence.

(1) Nucleic Acids and Coding Sequences Encoding Proteins

Provided herein are coding sequences of proteins capable of carrying out a biologic function. Coding sequences encoding the proteins set forth herein may be generated using routine methods. Also described herein are isolated nucleic acids comprising nucleic acid sequences that encode proteins.

In one embodiment, coding sequences of antigens capable of eliciting an immune response are provided. The antigen may contain at least one antigenic epitope that may be effective against particular immunogens against which an immune response can be induced. The nucleic acid sequences may optionally comprise coding sequences that encode a signal peptide such as for example an IgE or IgG signal peptide.

The nucleic acid sequence may encode a full-length protein. For example, the nucleic acid sequence may encode a full-length immunoadhesin protein. In one embodiment, the nucleic acid sequence may comprise a sequence that encodes SEQ ID NO:3, a variant thereof, or a fragment thereof. In one embodiment, the nucleic acid sequence comprises an RNA sequence encoding a full-length protein. For example, nucleic acids may comprise an RNA sequence encoding an immunoadhesin. In one embodiment, nucleic acids may comprise an RNA sequence encoding an eCD4-Ig. In one embodiment, nucleic acids may comprise an RNA sequence encoding more of SEQ ID NOs: 3, a variant thereof, a fragment thereof or any combination thereof.

The nucleic acid sequence may encode a fragment of a protein. Fragments of a full-length proteins can comprise at least 10%, at least 20%, at least 30%, at least 40%, at least 50%, at least 60%, at least 70%, at least 80%, at least 90%, or at least 95% of one or more of the full-length protein. For example, fragments of a nucleic acid encoding eCD4-Ig can comprise at least 10%, at least 20%, at least 30%, at least 40%, at least 50%, at least 60%, at least 70%, at least 80%, at least 90%, or at least 95% of one or more of the nucleic acid sequences set forth herein.

The nucleic acid sequence may encode a protein homologous to a biologic protein. For example, the nucleic acid sequence may encode a protein homologous to eCD4-Ig. Nucleic acid sequence may comprise a sequence that encodes a protein homologous to SEQ ID NOs: 3.

b. Antigen

Provided herein are immunogenic composition capable of eliciting an immune response. The proteins can treat, prevent, and/or protect against disease or infection, in the subject administered a composition of the invention. In one embodiment, the immunogenic composition is an antigen capable of eliciting an immune response in a mammal.

In one embodiment, the immunogenic composition can also comprise an antigen, or fragment or variant thereof. The antigen can be anything that induces an immune response in a subject. The antigen can be a nucleic acid sequence, an amino acid sequence, or a combination thereof. The nucleic acid sequence can be DNA, RNA, cDNA, a variant thereof, a fragment thereof, or a combination thereof. The nucleic acid sequence can also include additional sequences that encode linker or tag sequences that are linked to the antigen by a peptide bond. The amino acid sequence can be a protein, a peptide, a variant thereof, a fragment thereof, or a combination thereof.

The antigen can be contained in a protein, a nucleic acid, or a fragment thereof, or a variant thereof, or a combination thereof from any number of organisms, for example, a virus, a parasite, a bacterium, a fungus, or a mammal. The antigen can be associated with an autoimmune disease, allergy, or asthma. In other embodiments, the antigen can be associated with cancer, herpes, influenza, hepatitis B, hepatitis C, human papilloma virus (HPV), or human immunodeficiency virus (HIV).

(1) Viral Antigens

The antigen can be a viral antigen, or fragment thereof, or variant thereof. The viral antigen can be from a virus from one of the following families: Adenoviridae, Arenaviridae, Bunyaviridae, Caliciviridae, Coronaviridae, Filoviridae, Hepadnaviridae, Herpesviridae, Orthomyxoviridae, Papovaviridae, Paramyxoviridae, Parvoviridae, Picornaviridae, Poxviridae, Polyomaviridae, Reoviridae, Retroviridae, Rhabdoviridae, or Togaviridae. The viral antigen can be from papilloma viruses, for example, human papilloma virus (HPV), human immunodeficiency virus (HIV), polio virus, hepatitis viruses, for example, hepatitis A virus (HAV), hepatitis B virus (HBV), hepatitis C virus (HCV), hepatitis D virus (HDV), and hepatitis E virus (HEV), human T-cell leukemia virus (HTLV-I), hairy cell leukemia virus (HTLV-II), herpes simplex 1 (HSV1; oral herpes), herpes simplex 2 (HSV2; genital herpes), herpes zoster (VZV; varicella-zoster, a.k.a., chickenpox), Epstein-Barr virus (EBV), Merkel cell polyoma virus (MCV), or cancer causing virus.

(a) Hepatitis Antigen

The hepatitis antigen can be an antigen or immunogen from hepatitis A virus (HAV), hepatitis B virus (HBV), hepatitis C virus (HCV), hepatitis D virus (HDV), and/or hepatitis E virus (HEV). In some embodiments, the hepatitis antigen can be a nucleic acid molecule(s), such as a plasmid(s), which encodes one or more of the antigens from HAV, HBV, HCV, HDV, and HEV. The hepatitis antigen can be full-length or immunogenic fragments of full-length proteins.

The hepatitis antigen can comprise consensus sequences and/or modification for improved expression. Genetic modifications including codon optimization, RNA optimization, and the addition of a high efficient immunoglobulin leader sequence to increase the immunogenicity of the constructs can be included in the modified consensus sequences. The consensus hepatitis antigen may comprise a signal peptide such as an immunoglobulin signal peptide such as an IgE or IgG signal peptide, and in some embodiments, may comprise an HA tag. The immunogens can be designed to elicit stronger and broader cellular immune responses than corresponding codon optimized immunogens.

The hepatitis antigen can be an antigen from HAV. The hepatitis antigen can be a HAV capsid protein, a HAV non-structural protein, a fragment thereof, a variant thereof, or a combination thereof.

The hepatitis antigen can be an antigen from HCV. The hepatitis antigen can be a HCV nucleocapsid protein (i.e., core protein), a HCV envelope protein (e.g., E1 and E2), a HCV non-structural protein (e.g., NS1, NS2, NS3, NS4a, NS4b, NS5a, and NS5b), a fragment thereof, a variant thereof, or a combination thereof.

The hepatitis antigen can be an antigen from HDV. The hepatitis antigen can be a HDV delta antigen, fragment thereof, or variant thereof.

The hepatitis antigen can be an antigen from HEV. The hepatitis antigen can be a HEV capsid protein, fragment thereof, or variant thereof.

The hepatitis antigen can be an antigen from HBV. The hepatitis antigen can be a HBV core protein, a HBV surface protein, a HBV DNA polymerase, a HBV protein encoded by gene X, fragment thereof, variant thereof, or combination thereof. The hepatitis antigen can be a HBV genotype A core protein, a HBV genotype B core protein, a HBV genotype C core protein, a HBV genotype D core protein, a HBV genotype E core protein, a HBV genotype F core protein, a HBV genotype G core protein, a HBV genotype H core protein, a HBV genotype A surface protein, a HBV genotype B surface protein, a HBV genotype C surface protein, a HBV genotype D surface protein, a HBV genotype E surface protein, a HBV genotype F surface protein, a HBV genotype G surface protein, a HBV genotype H surface protein, fragment thereof, variant thereof, or combination thereof. The hepatitis antigen can be a consensus HBV core protein, or a consensus HBV surface protein.

In some embodiments, the hepatitis antigen can be a HBV genotype A consensus core DNA sequence construct, an IgE leader sequence linked to a consensus sequence for HBV genotype A core protein, or a HBV genotype A consensus core protein sequence.

In other embodiments, the hepatitis antigen can be a HBV genotype B consensus core DNA sequence construct, an IgE leader sequence linked to a consensus sequence for HBV genotype B core protein, or a HBV genotype B consensus core protein sequence.

In still other embodiments, the hepatitis antigen can be a HBV genotype C consensus core DNA sequence construct, an IgE leader sequence linked to a consensus sequence for HBV genotype C core protein, or a HBV genotype C consensus core protein sequence.

In some embodiments, the hepatitis antigen can be a HBV genotype D consensus core DNA sequence construct, an IgE leader sequence linked to a consensus sequence for HBV genotype D core protein, or a HBV genotype D consensus core protein sequence.

In other embodiments, the hepatitis antigen can be a HBV genotype E consensus core DNA sequence construct, an IgE leader sequence linked to a consensus sequence for HBV genotype E core protein, or a HBV genotype E consensus core protein sequence.

In some embodiments, the hepatitis antigen can be a HBV genotype F consensus core DNA sequence construct, an IgE leader sequence linked to a consensus sequence for HBV genotype F core protein, or a HBV genotype F consensus core protein sequence.

In other embodiments, the hepatitis antigen can be a HBV genotype G consensus core DNA sequence construct, an IgE leader sequence linked to a consensus sequence for HBV genotype G core protein, or a HBV genotype G consensus core protein sequence.

In some embodiments, the hepatitis antigen can be a HBV genotype H consensus core DNA sequence construct, an IgE leader sequence linked to a consensus sequence for HBV genotype H core protein, or a HBV genotype H consensus core protein sequence.

In still other embodiments, the hepatitis antigen can be a HBV genotype A consensus surface DNA sequence construct, an IgE leader sequence linked to a consensus sequence for HBV genotype A surface protein, or a HBV genotype A consensus surface protein sequence.

In some embodiments, the hepatitis antigen can be a HBV genotype B consensus surface DNA sequence construct, an IgE leader sequence linked to a consensus sequence for HBV genotype B surface protein, or a HBV genotype B consensus surface protein sequence.

In other embodiments, the hepatitis antigen can be a HBV genotype C consensus surface DNA sequence construct, an IgE leader sequence linked to a consensus sequence for HBV genotype C surface protein, or a HBV genotype C consensus surface protein sequence.

In still other embodiments, the hepatitis antigen can be a HBV genotype D consensus surface DNA sequence construct, an IgE leader sequence linked to a consensus sequence for HBV genotype D surface protein, or a HBV genotype D consensus surface protein sequence.

In some embodiments, the hepatitis antigen can be a HBV genotype E consensus surface DNA sequence construct, an IgE leader sequence linked to a consensus sequence for HBV genotype E surface protein, or a HBV genotype E consensus surface protein sequence.

In other embodiments, the hepatitis antigen can be a HBV genotype F consensus surface DNA sequence construct, an IgE leader sequence linked to a consensus sequence for HBV genotype F surface protein, or a HBV genotype F consensus surface protein sequence.

In still other embodiments, the hepatitis antigen can be a HBV genotype G consensus surface DNA sequence construct, an IgE leader sequence linked to a consensus sequence for HBV genotype G surface protein, or a HBV genotype G consensus surface protein sequence.

In other embodiments, the hepatitis antigen can be a HBV genotype H consensus surface DNA sequence construct, an IgE leader sequence linked to a consensus sequence for HBV genotype H surface protein, or a HBV genotype H consensus surface protein sequence.

(b) Human Papilloma Virus (HPV) Antigen

The HPV antigen can be from HPV types 16, 18, 31, 33, 35, 45, 52, and 58 which cause cervical cancer, rectal cancer, and/or other cancers. The HPV antigen can be from HPV types 6 and 11, which cause genital warts, and are known to be causes of head and neck cancer.

The HPV antigens can be the HPV E6 or E7 domains from each HPV type. For example, for HPV type 16 (HPV16), the HPV16 antigen can include the HPV16 E6 antigen, the HPV16 E7 antigen, fragments, variants, or combinations thereof. Similarly, the HPV antigen can be HPV 6 E6 and/or E7, HPV 11 E6 and/or E7, HPV 18 E6 and/or E7, HPV 31 E6 and/or E7, HPV 33 E6 and/or E7, HPV 52 E6 and/or E7, or HPV 58 E6 and/or E7, fragments, variants, or combinations thereof.

(c) RSV Antigen

The RSV antigen can be a human RSV fusion protein (also referred to herein as “RSV F”, “RSV F protein” and “F protein”), or fragment or variant thereof. The human RSV fusion protein can be conserved between RSV subtypes A and B. The RSV antigen can be a RSV F protein, or fragment or variant thereof, from the RSV Long strain (GenBank AAX23994.1). The RSV antigen can be a RSV F protein from the RSV A2 strain (GenBank AAB59858.1), or a fragment or variant thereof. The RSV antigen can be a monomer, a dimer or trimer of the RSV F protein, or a fragment or variant thereof. The RSV antigen can be an optimized amino acid RSV F amino acid sequence, or fragment or variant thereof.

The post-fusion form of RSV F elicits high titer neutralizing antibodies in immunized animals and protects the animals from RSV challenge. The present invention utilizes this immune response in the claimed vaccines. According to the invention, the RSV F protein can be in a prefusion form or a postfusion form.

The RSV antigen can also be human RSV attachment glycoprotein (also referred to herein as “RSV G”, “RSV G protein” and “G protein”), or fragment or variant thereof. The human RSV G protein differs between RSV subtypes A and B. The antigen can be RSV G protein, or fragment or variant thereof, from the RSV Long strain (GenBank AAX23993). The RSV antigen can be RSV G protein from: the RSV subtype B isolate H5601, the RSV subtype B isolate H1068, the RSV subtype B isolate H5598, the RSV subtype B isolate H1123, or a fragment or variant thereof. The RSV antigen can be an optimized amino acid RSV G amino acid sequence, or fragment or variant thereof.

In other embodiments, the RSV antigen can be human RSV non-structural protein 1 (“NS1 protein”), or fragment or variant thereof. For example, the RSV antigen can be RSV NS1 protein, or fragment or variant thereof, from the RSV Long strain (GenBank AAX23987.1). The RSV antigen human can also be RSV non-structural protein 2 (“NS2 protein”), or fragment or variant thereof. For example, the RSV antigen can be RSV NS2 protein, or fragment or variant thereof, from the RSV Long strain (GenBank AAX23988.1). The RSV antigen can further be human RSV nucleocapsid (“N”) protein, or fragment or variant thereof. For example, the RSV antigen can be RSV N protein, or fragment or variant thereof, from the RSV Long strain (GenBank AAX23989.1). The RSV antigen can be human RSV Phosphoprotein (“P”) protein, or fragment or variant thereof. For example, the RSV antigen can be RSV P protein, or fragment or variant thereof, from the RSV Long strain (GenBank AAX23990.1). The RSV antigen also can be human RSV Matrix protein (“M”) protein, or fragment or variant thereof. For example, the RSV antigen can be RSV M protein, or fragment or variant thereof, from the RSV Long strain (GenBank AAX23991.1).

In still other embodiments, the RSV antigen can be human RSV small hydrophobic (“SH”) protein, or fragment or variant thereof. For example, the RSV antigen can be RSV SH protein, or fragment or variant thereof, from the RSV Long strain (GenBank AAX23992.1). The RSV antigen can also be human RSV Matrix protein 2-1 (“M2-1”) protein, or fragment or variant thereof. For example, the RSV antigen can be RSV M2-1 protein, or fragment or variant thereof, from the RSV Long strain (GenBank AAX23995.1). The RSV antigen can further be human RSV Matrix protein 2-2 (“M2-2”) protein, or fragment or variant thereof. For example, the RSV antigen can be RSV M2-2 protein, or fragment or variant thereof, from the RSV Long strain (GenBank AAX23997.1). The RSV antigen human can be RSV Polymerase L (“L”) protein, or fragment or variant thereof. For example, the RSV antigen can be RSV L protein, or fragment or variant thereof, from the RSV Long strain (GenBank AAX23996.1).

In further embodiments, the RSV antigen can have an optimized amino acid sequence of NS1, NS2, N, P, M, SH, M2-1, M2-2, or L protein. The RSV antigen can be a human RSV protein or recombinant antigen, such as any one of the proteins encoded by the human RSV genome.

In other embodiments, the RSV antigen can be, but is not limited to, the RSV F protein from the RSV Long strain, the RSV G protein from the RSV Long strain, the optimized amino acid RSV G amino acid sequence, the human RSV genome of the RSV Long strain, the optimized amino acid RSV F amino acid sequence, the RSV NS1 protein from the RSV Long strain, the RSV NS2 protein from the RSV Long strain, the RSV N protein from the RSV Long strain, the RSV P protein from the RSV Long strain, the RSV M protein from the RSV Long strain, the RSV SH protein from the RSV Long strain, the RSV M2-1 protein from the RSV Long strain, the RSV M2-2 protein from the RSV Long strain, the RSV L protein from the RSV Long strain, the RSV G protein from the RSV subtype B isolate H5601, the RSV G protein from the RSV subtype B isolate H1068, the RSV G protein from the RSV subtype B isolate H5598, the RSV G protein from the RSV subtype B isolate H1123, or fragment thereof, or variant thereof.

(d) Influenza Antigen

The influenza antigens are those capable of eliciting an immune response in a mammal against one or more influenza serotypes. The antigen can comprise the full length translation product HA0, subunit HA1, subunit HA2, a variant thereof, a fragment thereof or a combination thereof. The influenza hemagglutinin antigen can be a consensus sequence derived from multiple strains of influenza A serotype H1, a consensus sequence derived from multiple strains of influenza A serotype H2, a hybrid sequence containing portions of two different consensus sequences derived from different sets of multiple strains of influenza A serotype H1 or a consensus sequence derived from multiple strains of influenza B. The influenza hemagglutinin antigen can be from influenza B.

The influenza antigen can also contain at least one antigenic epitope that can be effective against particular influenza immunogens against which an immune response can be induced. The antigen may provide an entire repertoire of immunogenic sites and epitopes present in an intact influenza virus. The antigen may be a consensus hemagglutinin antigen sequence that can be derived from hemagglutinin antigen sequences from a plurality of influenza A virus strains of one serotype such as a plurality of influenza A virus strains of serotype H1 or of serotype H2. The antigen may be a hybrid consensus hemagglutinin antigen sequence that can be derived from combining two different consensus hemagglutinin antigen sequences or portions thereof. Each of two different consensus hemagglutinin antigen sequences may be derived from a different set of a plurality of influenza A virus strains of one serotype such as a plurality of influenza A virus strains of serotype H1. The antigen may be a consensus hemagglutinin antigen sequence that can be derived from hemagglutinin antigen sequences from a plurality of influenza B virus strains.

In some embodiments, the influenza antigen can be H1 HA, H2 HA, H3 HA, H5 HA, or a BHA antigen. Alternatively, the influenza antigen can be a consensus hemagglutinin antigen comprising a consensus H1 amino acid sequence or a consensus H2 amino acid sequence. The consensus hemagglutinin antigen may be a synthetic hybrid consensus H1 sequence comprising portions of two different consensus H1 sequences, which are each derived from a different set of sequences from the other. An example of a consensus HA antigen that is a synthetic hybrid consensus H1 protein is a protein comprising the U2 amino acid sequence. The consensus hemagglutinin antigen may be a consensus hemagglutinin protein derived from hemagglutinin sequences from influenza B strains, such as a protein comprising the consensus BHA amino acid sequence.

The consensus hemagglutinin antigen may further comprise one or more additional amino acid sequence elements. The consensus hemagglutinin antigen may further comprise on its N-terminal an IgE or IgG leader amino acid sequence. The consensus hemagglutinin antigen may further comprise an immunogenic tag which is a unique immunogenic epitope that can be detected by readily available antibodies. An example of such an immunogenic tag is the 9 amino acid influenza HA Tag which may be linked on the consensus hemagglutinin C terminus. In some embodiments, consensus hemagglutinin antigen may further comprise on its N-terminal an IgE or IgG leader amino acid sequence and on its C terminal an HA tag.

The consensus hemagglutinin antigen may be a consensus hemagglutinin protein that consists of consensus influenza amino acid sequences or fragments and variants thereof. The consensus hemagglutinin antigen may be a consensus hemagglutinin protein that comprises non-influenza protein sequences and influenza protein sequences or fragments and variants thereof.

Examples of a consensus H1 protein include those that may consist of the consensus H1 amino acid sequence or those that further comprise additional elements such as an IgE leader sequence, or an HA Tag or both an IgE leader sequence and an HA Tag.

Examples of consensus H2 proteins include those that may consist of the consensus H2 amino acid sequence or those that further comprise an IgE leader sequence, or an HA Tag, or both an IgE leader sequence and an HA Tag.

Examples of hybrid consensus H1 proteins include those that may consist of the consensus U2 amino acid sequence or those that further comprise an IgE leader sequence, or an HA Tag, or both an IgE leader sequence and an HA Tag.

Examples of hybrid consensus influenza B hemagglutinin proteins include those that may consist of the consensus BHA amino acid sequence or it may comprise an IgE leader sequence, or an HA Tag, or both an IgE leader sequence and an HA Tag.

The consensus hemagglutinin protein can be encoded by a consensus hemagglutinin nucleic acid, a variant thereof or a fragment thereof. Unlike the consensus hemagglutinin protein which may be a consensus sequence derived from a plurality of different hemagglutinin sequences from different strains and variants, the consensus hemagglutinin nucleic acid refers to a nucleic acid sequence that encodes a consensus protein sequence and the coding sequences used may differ from those used to encode the particular amino acid sequences in the plurality of different hemagglutinin sequences from which the consensus hemagglutinin protein sequence is derived. The consensus nucleic acid sequence may be codon optimized and/or RNA optimized. The consensus hemagglutinin nucleic acid sequence may comprise a Kozak's sequence in the 5′ untranslated region. The consensus hemagglutinin nucleic acid sequence may comprise nucleic acid sequences that encode a leader sequence. The coding sequence of an N terminal leader sequence is 5′ of the hemagglutinin coding sequence. The N-terminal leader can facilitate secretion. The N-terminal leader can be an IgE leader or an IgG leader. The consensus hemagglutinin nucleic acid sequence can comprise nucleic acid sequences that encode an immunogenic tag. The immunogenic tag can be on the C terminus of the protein and the sequence encoding it is 3′ of the HA coding sequence. The immunogenic tag provides a unique epitope for which there are readily available antibodies so that such antibodies can be used in assays to detect and confirm expression of the protein. The immunogenic tag can be an H Tag at the C-terminus of the protein.

(e) Human Immunodeficiency Virus (HIV) Antigen

HIV antigens can include modified consensus sequences for immunogens. Genetic modifications including codon optimization, RNA optimization, and the addition of a high efficient immunoglobin leader sequence to increase the immunogenicity of constructs can be included in the modified consensus sequences. The novel immunogens can be designed to elicit stronger and broader cellular immune responses than a corresponding codon optimized immunogens.

In some embodiments, the HIV antigen can be a subtype A consensus envelope DNA sequence construct, an IgE leader sequence linked to a consensus sequence for Subtype A envelope protein, or a subtype A consensus Envelope protein sequence.

In other embodiments, the HIV antigen can be a subtype B consensus envelope DNA sequence construct, an IgE leader sequence linked to a consensus sequence for Subtype B envelope protein, or a subtype B consensus Envelope protein sequence.

In still other embodiments, the HIV antigen can be a subtype C consensus envelope DNA sequence construct, an IgE leader sequence linked to a consensus sequence for subtype C envelope protein, or a subtype C consensus envelope protein sequence.

In further embodiments, the HIV antigen can be a subtype D consensus envelope DNA sequence construct, an IgE leader sequence linked to a consensus sequence for Subtype D envelope protein, or a subtype D consensus envelope protein sequence.

In some embodiments, the HIV antigen can be a subtype B Nef-Rev consensus envelope DNA sequence construct, an IgE leader sequence linked to a consensus sequence for Subtype B Nef-Rev protein, or a Subtype B Nef-Rev consensus protein sequence.

In other embodiments, the HIV antigen can be a Gag consensus DNA sequence of subtype A, B, C and D DNA sequence construct, an IgE leader sequence linked to a consensus sequence for Gag consensus subtype A, B, C and D protein, or a consensus Gag subtype A, B, C and D protein sequence.

In still other embodiments the HIV antigen can be a Pol DNA sequence or a Pol protein sequence. The HIV antigen can be nucleic acid or amino acid sequences of Env A, Env B, Env C, Env D, B Nef-Rev, Gag, or any combination thereof.

(f) Herpes Antigen

In one embodiment, the herpes antigen is from HCMV, HSV1, HSV2, CeHV1, VZV or EBV. The herpes antigens comprise immunogenic proteins including gB, gM, gN, gH, gL, gO, gE, gI, gK, gC, gD, UL128, UL130, UL-131A, UL-83 (pp65), whether from HCMV, HSV1, HSV2, CeHV1, VZV or EBV. In some embodiments, the antigens can be HSV1-gH, HSV1-gL, HSV1-gC, HSV1-gD, HSV2-gH, HSV2-gL, HSV2-gC, HSV2-gD, VZV-gH, VZV-gL, VZV-gM, VZV-gN, CeHV1-gH, CeHV1-gL, CeHV1-gC, CeHV1-gD, VZV-gE, or VZV-gI.

(2) Parasite Antigen

In one embodiment, the parasite can be a protozoa, helminth, or ectoparasite. The helminth (i.e., worm) can be a flatworm (e.g., flukes and tapeworms), a thorny-headed worm, or a round worm (e.g., pinworms). The ectoparasite can be lice, fleas, ticks, and mites.

The parasite can be any parasite causing the following diseases: Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Malaria, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, and Trichuriasis.

The parasite can be Acanthamoeba, Anisakis, Ascaris lumbricoides, Botfly, Balantidium coli, Bedbug, Cestoda (tapeworm), Chiggers, Cochliomyia hominivorax, Entamoeba histolytica, Fasciola hepatica, Giardia lamblia, Hookworm, Leishmania, Linguatula serrata, Liver fluke, Loa loa, Paragonimus—lung fluke, Pinworm, Plasmodium falciparum, Schistosoma, Strongyloides stercoralis, Mite, Tapeworm, Toxoplasma gondii, Trypanosoma, Whipworm, or Wuchereria bancrofti.

(a) Malaria Antigen

In one embodiment, the antigen can be from a parasite causing malaria. The malaria causing parasite can be Plasmodium falciparum. The Plasmodium falciparum antigen can include the circumsporozoite (CS) antigen.

In some embodiments, the malaria antigen can be nucleic acid molecules such as plasmids which encode one or more of the P. falciparum immunogens CS; LSA1; TRAP; CelTOS; and Ama1. The immunogens may be full length or immunogenic fragments of full length proteins. The immunogens comprise consensus sequences and/or modifications for improved expression.

In other embodiments, the malaria antigen can be a consensus sequence of TRAP, which is also referred to as SSP2, designed from a compilation of all full-length Plasmodium falciparum TRAP/SSP2 sequences in the GenBank database (28 sequences total). Consensus TRAP immunogens (i.e., ConTRAP immunogen) may comprise a signal peptide such as an immunoglobulin signal peptide such as an IgE or IgG signal peptide and in some embodiments, may comprise an HA Tag.

In still other embodiments, the malaria antigen can be CelTOS, which is also referred to as Ag2 and is a highly conserved Plasmodium antigen. Consensus CelTOS antigens (i.e., ConCelTOS immunogen) may comprise a signal peptide such as an immunoglobulin signal peptide such as an IgE or IgG signal peptide and in some embodiments, may comprise an HA Tag.

In further embodiments, the malaria antigen can be Ama1, which is a highly conserved Plasmodium antigen. The malaria antigen can also be a consensus sequence of Ama1 (i.e., ConAmaI immunogen) comprising in some instances, a signal peptide such as an immunoglobulin signal peptide such as an IgE or IgG signal peptide and in some embodiments, may comprise an HA Tag.

In some embodiments, the malaria antigen can be a consensus CS antigen (i.e., Consensus CS immunogen) comprising in some instances, a signal peptide such as an immunoglobulin signal peptide such as an IgE or IgG signal peptide and in some embodiments, may comprise an HA Tag.

In other embodiments, the malaria antigen can be a fusion protein comprising a combination of two or more of the PF proteins set forth herein. For example, fusion proteins may comprise two or more of Consensus CS immunogen, ConLSA1 immunogen, ConTRAP immunogen, ConCelTOS immunogen and ConAma1 immunogen linked directly adjacent to each other or linked with a spacer or one or more amino acids in between. In some embodiments, the fusion protein comprises two PF immunogens; in some embodiments the fusion protein comprises three PF immunogens, in some embodiments the fusion protein comprises four PF immunogens, and in some embodiments the fusion protein comprises five PF immunogens. Fusion proteins with two Consensus PF immunogens may comprise: CS and LSA1; CS and TRAP; CS and CelTOS; CS and Ama1; LSA1 and TRAP; LSA1 and CelTOS; LSA1 and Ama1; TRAP and CelTOS; TRAP and Ama1; or CelTOS and Ama1. Fusion proteins with three Consensus PF immunogens may comprise: CS, LSA1 and TRAP; CS, LSA1 and CelTOS; CS, LSA1 and Ama1; LSA1, TRAP and CelTOS; LSA1, TRAP and Ama1; or TRAP, CelTOS and Ama1. Fusion proteins with four Consensus PF immunogens may comprise: CS, LSA1, TRAP and CelTOS; CS, LSA1, TRAP and Ama1; CS, LSA1, CelTOS and Ama1; CS, TRAP, CelTOS and Ama1; or LSA1, TRAP, CelTOS and Ama1. Fusion proteins with five Consensus PF immunogens may comprise CS or CS-alt, LSA1, TRAP, CelTOS and Ama1.

In some embodiments, the fusion proteins comprise a signal peptide linked to the N terminus. In some embodiments, the fusion proteins comprise multiple signal peptides linked to the N terminal of each Consensus PF immunogen. In some embodiments, a spacer may be included between PF immunogens of a fusion protein. In some embodiments, the spacer between PF immunogens of a fusion protein may be a proteolyic cleavage site. In some embodiments, the spacer may be a proteolyic cleavage site recognized by a protease found in cells to which the immunogenic composition is intended to be administered and/or taken up. In some embodiments, a spacer may be included between PF immunogens of a fusion protein wherein the spacer is a proteolyic cleavage site recognized by a protease found in cells to which the immunogenic composition is intended to be administered and/or taken up and the fusion proteins comprises multiple signal peptides linked to the N terminal of each Consensus PF immunogens such that upon cleavage the signal peptide of each Consensus PF immunogens translocates the Consensus PF immunogen to outside the cell.

(3) Bacterial Antigens

In one embodiment, the bacterium can be from any one of the following phyla: Acidobacteria, Actinobacteria, Aquificae, Bacteroidetes, Caldiserica, Chlamydiae, Chlorobi, Chloroflexi, Chrysiogenetes, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospira, Planctomycetes, Proteobacteria, Spirochaetes, Synergistetes, Tenericutes, Thermodesulfobacteria, Thermotogae, and Verrucomicrobia.

The bacterium can be a gram positive bacterium or a gram negative bacterium. The bacterium can be an aerobic bacterium or an anerobic bacterium. The bacterium can be an autotrophic bacterium or a heterotrophic bacterium. The bacterium can be a mesophile, a neutrophile, an extremophile, an acidophile, an alkaliphile, a thermophile, a psychrophile, an halophile, or an osmophile.

The bacterium can be an anthrax bacterium, an antibiotic resistant bacterium, a disease causing bacterium, a food poisoning bacterium, an infectious bacterium, Salmonella bacterium, Staphylococcus bacterium, Streptococcus bacterium, or tetanus bacterium. The bacterium can be a mycobacteria, Clostridium tetani, Yersinia pestis, Bacillus anthracis, methicillin-resistant Staphylococcus aureus (MRSA), or Clostridium difficile. The bacterium can be Mycobacterium tuberculosis.

(a) Mycobacterium tuberculosis Antigens

In one embodiment, the TB antigen can be from the Ag85 family of TB antigens, for example, Ag85A and Ag85B. The TB antigen can be from the Esx family of TB antigens, for example, EsxA, EsxB, EsxC, EsxD, EsxE, EsxF, EsxH, EsxO, EsxQ, EsxR, EsxS, EsxT, EsxU, EsxV, and EsxW. The TB antigen can include resuscitation factors RpfA, RpfB, and RpfD. The TB antigens can also include RV1733c, ESAT6, PPE51, RV2626c, RV2628, RV2034, Rv0995, RV0990c, RV0012, RV1872c, RV0010c, RV2719c and RV3407.

In some embodiments, the TB antigen can be nucleic acid molecules such as plasmids which encode one or more of the Mycobacterium tuberculosis immunogens from the Ag85 family and the Esx family. The immunogens can be full-length or immunogenic fragments of full-length proteins. The immunogens can comprise consensus sequences and/or modifications for improved expression. Consensus immunogens may comprise a signal peptide such as an immunoglobulin signal peptide such as an IgE or IgG signal peptide and in some embodiments, may comprise an HA tag.

(4) Fungal Antigens

In one embodiment, the fungus can be Aspergillus species, Blastomyces dermatitidis, Candida yeasts (e.g., Candida albicans), Coccidioides, Cryptococcus neoformans, Cryptococcus gattii, dermatophyte, Fusarium species, Histoplasma capsulatum, Mucoromycotina, Pneumocystis jirovecii, Sporothrix schenckii, Exserohilum, or Cladosporium.

(5) Tumor Antigen

In the context of the present invention, “tumor antigen” or “hyperproliferative disorder antigen” or “antigen associated with a hyperproliferative disorder,” refers to antigens that are common to specific hyperproliferative disorders such as cancer. The antigens discussed herein are merely included by way of example. The list is not intended to be exclusive and further examples will be readily apparent to those of skill in the art.

Tumor antigens are proteins that are produced by tumor cells that elicit an immune response, particularly T-cell mediated immune responses. The selection of the antigen binding moiety of the invention will depend on the particular type of cancer to be treated. Tumor antigens are well known in the art and include, for example, a glioma-associated antigen, carcinoembryonic antigen (CEA), (3-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In one embodiment, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The type of tumor antigen referred to in the invention may also be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

Cancer markers are known proteins that are present or upregulated vis-à-vis certain cancer cells. By methodology of generating antigens that represent such markers in a way to break tolerance to self, a cancer vaccine can be generated. Such cancer vaccines can include the CTLA4 antibody and optionally one or more antibody targeting one or more additional immune checkpoint proteins to enhance the immune response. The following are some exemplary tumor antigens:

(a) TERT

TERT is a telomerase reverse transcriptase that synthesizes a TTAGGG tag on the end of telomeres to prevent cell death due to chromosomal shortening. Hyperproliferative cells with abnormally high expression of TERT may be targeted by immunotherapy. Recent studies demonstrate that TERT expression in dendritic cells transfected with TERT genes can induce CD8+ cytotoxic T cells and elicit a CD4+ T cells in an antigen-specific fashion.

(b) Prostate Antigens

The following are antigens capable of eliciting an immune response in a mammal against a prostate antigen. The consensus antigen can comprise epitopes that make them particularly effective as immunogens against prostate cancer cells can be induced. The consensus prostate antigen can comprise the full length translation product, a variant thereof, a fragment thereof or a combination thereof.

The prostate antigens can include one or more of the following: PSA antigen, PSMA antigen, STEAP antigen, PSCA antigen, Prostatic acid phosphatase (PAP) antigen, and other known prostate tumor antigens. Proteins may comprise sequences homologous to the prostate antigens, fragments of the prostate antigens and proteins with sequences homologous to fragments of the prostate antigens.

(c) WT1

The antigen can be Wilm's tumor suppressor gene 1 (WT1), a fragment thereof, a variant thereof, or a combination thereof. WT1 is a transcription factor containing at the N-terminus, a proline/glutamine-rich DNA-binding domain and at the C-terminus, four zinc finger motifs. WT1 plays a role in the normal development of the urogenital system and interacts with numerous factors, for example, p53, a known tumor suppressor and the serine protease HtrA2, which cleaves WT1 at multiple sites after treatment with a cytotoxic drug.

Mutation of WT1 can lead to tumor or cancer formation, for example, Wilm's tumor or tumors expressing WT1. Wilm's tumor often forms in one or both kidneys before metastasizing to other tissues, for example, but not limited to, liver tissue, urinary tract system tissue, lymph tissue, and lung tissue. Accordingly, Wilm's tumor can be considered a metastatic tumor. Wilm's tumor usually occurs in younger children (e.g., less than 5 years old) and in both sporadic and hereditary forms. Accordingly, the immunogenic composition can be used for treating subjects suffering from Wilm's tumor. The immunogenic composition can also be used for treating subjects with cancers or tumors that express WT1 for preventing development of such tumors in subjects. The WT1 antigen can differ from the native, “normal” WT1 gene, and thus, provide therapy or prophylaxis against an WT1 antigen-expressing tumor. Proteins may comprise sequences homologous to the WT1 antigens, fragments of the WT1 antigens and proteins with sequences homologous to fragments of the WT1 antigens.

(d) Tyrosinase Antigen

The antigen tyrosinase (Tyr) antigen is an important target for immune mediated clearance by inducing (1) humoral immunity via B cell responses to generate antibodies that block monocyte chemoattractant protein-1 (MCP-1) production, thereby retarding myeloid derived suppressor cells (MDSCs) and suppressing tumor growth; (2) increase cytotoxic T lymphocyte such as CD8⁺ (CTL) to attack and kill tumor cells; (3) increase T helper cell responses; (4) and increase inflammatory responses via IFN-γ and TFN-α or all of the aforementioned.

Tyrosinase is a copper-containing enzyme that can be found in plant and animal tissues. Tyrosinase catalyzes the production of melanin and other pigments by the oxidation of phenols such as tyrosine. In melanoma, tyrosinase can become unregulated, resulting in increased melanin synthesis. Tyrosinase is also a target of cytotoxic T cell recognition in subjects suffering from melanoma. Accordingly, tyrosinase can be an antigen associated with melanoma.

The antigen can comprise protein epitopes that make them particularly effective as immunogens against which anti-Tyr immune responses can be induced. The Tyr antigen can comprise the full length translation product, a variant thereof, a fragment thereof or a combination thereof.

The Tyr antigen can comprise a consensus protein. The Tyr antigen induces antigen-specific T-cell and high titer antibody responses both systemically against all cancer and tumor related cells. As such, a protective immune response is provided against tumor formation by vaccines comprising the Tyr consensus antigen. Accordingly, any user can design an immunogenic composition of the present invention to include a Tyr antigen to provide broad immunity against tumor formation, metastasis of tumors, and tumor growth. Proteins may comprise sequences homologous to the Tyr antigens, fragments of the Tyr antigens and proteins with sequences homologous to fragments of the Tyr antigens.

(e) NY-ESO-1

NY-ESO-1 is a cancer-testis antigen expressed in various cancers where it can induce both cellular and humoral immunity. Gene expression studies have shown upregulation of the gene for NY-ESO-1, CTAG1B, in myxoid and round cell liposarcomas.

In various embodiments, the NY-ESO-1 antigen comprises a consensus NY-ESO-1 protein or a nucleic acid molecule encoding a consensus NY-ESO-1 protein. NY-ESO-1 antigens include sequences homologous to the NY-ESO-1 antigens, fragments of the NY-ESO-1 antigens and proteins with sequences homologous to fragments of the NY-ESO-1 antigens.

(f) PRAME

Melanoma antigen preferentially expressed in tumors (PRAME antigen) is a protein that in humans is encoded by the PRAME gene. This gene encodes an antigen that is predominantly expressed in human melanomas and that is recognized by cytolytic T lymphocytes. It is not expressed in normal tissues, except testis. The gene is also expressed in acute leukemias. Five alternatively spliced transcript variants encoding the same protein have been observed for this gene. Proteins may comprise sequences homologous to the PRAME antigens, fragments of the PRAME antigens and proteins with sequences homologous to fragments of the PRAME antigens.

(g) MAGE

MAGE stands for Melanoma-associated Antigen, and in particular melanoma associated antigen 4 (MAGEA4). MAGE-A4 is expressed in male germ cells and tumor cells of various histological types such as gastrointestinal, esophageal and pulmonary carcinomas. MAGE-A4 binds the oncoprotein, Gankyrin. This MAGE-A4 specific binding is mediated by its C-terminus. Studies have shown that exogenous MAGE-A4 can partly inhibit the adhesion-independent growth of Gankyrin-overexpressing cells in vitro and suppress the formation of migrated tumors from these cells in nude mice. This inhibition is dependent upon binding between MAGE-A4 and Gankyrin, suggesting that interactions between Gankyrin and MAGE-A4 inhibit Gankyrin-mediated carcinogenesis. It is likely that MAGE expression in tumor tissue is not a cause, but a result of tumorgenesis, and MAGE genes take part in the immune process by targeting early tumor cells for destruction.

Melanoma-associated antigen 4 protein (MAGEA4) can be involved in embryonic development and tumor transformation and/or progression. MAGEA4 is normally expressed in testes and placenta. MAGEA4, however, can be expressed in many different types of tumors, for example, melanoma, head and neck squamous cell carcinoma, lung carcinoma, and breast carcinoma. Accordingly, MAGEA4 can be antigen associated with a variety of tumors.

The MAGEA4 antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune responses. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TFG-β, tumor associated macrophages, tumor associated fibroblasts.

The MAGEA4 antigen can comprise protein epitopes that make them particularly effective as immunogens against which anti-MAGEA4 immune responses can be induced. The MAGEA4 antigen can comprise the full length translation product, a variant thereof, a fragment thereof or a combination thereof. The MAGEA4 antigen can comprise a consensus protein.

The nucleic acid sequence encoding the consensus MAGEA4 antigen can be optimized with regards to codon usage and corresponding RNA transcripts. The nucleic acid encoding the consensus MAGEA4 antigen can be codon and RNA optimized for expression. In some embodiments, the nucleic acid sequence encoding the consensus MAGEA4 antigen can include a Kozak sequence (e.g., GCC ACC) to increase the efficiency of translation. The nucleic acid encoding the consensus MAGEA4 antigen can include multiple stop codons (e.g., TGA TGA) to increase the efficiency of translation termination.

(h) FSHR

Follicle stimulating hormone receptor (FSHR) is an antigen that is selectively expressed in women in the ovarian granulosa cells (Simoni et al., Endocr Rev. 1997, 18:739-773) and at low levels in the ovarian endothelium (Vannier et al., Biochemistry, 1996, 35:1358-1366). Most importantly, this surface antigen is expressed in 50-70% of ovarian carcinomas. In various embodiments, the FSHR antigen comprises a consensus protein or a nucleic acid molecule encoding a consensus protein. FSHR antigens include sequences homologous to the FSHR antigens, fragments of the FSHR antigens and proteins with sequences homologous to fragments of the FSHR antigens.

(i) Tumor Microenvironment Antigens

Several proteins are overexpressed in the tumor microenvironment including, but not limited to, Fibroblast Activation Protein (FAP), Platelet Derived Growth Factor Receptor Beta (PDGFR-β), and Glypican-1 (GPC1). FAP is a membrane-bound enzyme with gelatinase and peptidase activity that is up-regulated in cancer-associated fibroblasts in over 90% of human carcinomas. PDGFR-β is a cell surface tyrosine kinase receptor that has roles in the regulation of many biological processes including embryonic development, angiogenesis, cell proliferation and differentiation. GPC1 is a cell surface proteoglycan that is enriched in cancer cells.

c. Antibodies

Provided herein are antibodies that can bind or react with a desired antigen, which is described in more detail herein. The antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The antibody can treat, prevent, and/or protect against disease or infection, in the subject administered a composition of the invention. The antibody, by binding the antigen, can treat, prevent, and/or protect against disease or infection in the subject administered the composition. The antibody can promote survival of the disease in the subject administered the composition. In one embodiment, the antibody can provide increased survival of the disease in the subject over the expected survival of a subject having the disease who has not been administered the antibody. In various embodiments, the antibody can provide at least about a 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or a 100% increase in survival of the disease in subjects administered the composition over the expected survival in the absence of the composition. In one embodiment, the antibody can provide increased protection against the disease in the subject over the expected protection of a subject who has not been administered the antibody. In various embodiments, the antibody can protect against disease in at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% of subjects administered the composition over the expected protection in the absence of the composition.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment, which comprises both antigen-binding sites. Accordingly, the antibody can be the Fab or F(ab′)2. The Fab can include the heavy chain polypeptide and the light chain polypeptide. The heavy chain polypeptide of the Fab can include the VH region and the CH1 region. The light chain of the Fab can include the VL region and CL region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

The antibody can be a bispecific antibody as described herein in more detail. The antibody can be a bifunctional antibody as also described herein in more detail.

As described above, the antibody can be generated in the subject upon administration of the composition to the subject. The antibody may have a half-life within the subject. In some embodiments, the antibody may be modified to extend or shorten its half-life within the subject. Such modifications are described herein in more detail.

The antibody can be defucosylated as described in more detail herein.

The antibody may be modified to reduce or prevent antibody-dependent enhancement (ADE) of disease associated with the antigen as described in more detail herein.

(1) Nucleic Acid Synthetic Antibodies

Also provided herein are nucleic acid sequences antibodies for use for producing antibodies. In one embodiment, the antibodies can be produced in mammalian cells or for delivery in DNA or RNA vectors including bacterial, yeast, as well as viral vectors.

In one embodiment, the composition comprises a recombinant nucleic acid sequence encoding an antibody, a fragment thereof, a variant thereof, or a combination thereof. The composition, when administered to a subject in need thereof, can result in the generation of a synthetic antibody in the subject. The synthetic antibody can bind a target molecule (i.e., an antigen) present in the subject. Such binding can neutralize the antigen, block recognition of the antigen by another molecule, for example, a protein or nucleic acid, and elicit or induce an immune response to the antigen.

In one embodiment, the composition comprises a nucleotide sequence encoding a synthetic antibody. In one embodiment, the composition comprises a nucleic acid molecule comprising a first nucleotide sequence encoding a first synthetic antibody and a second nucleotide sequence encoding a second synthetic antibody. In one embodiment, the nucleic acid molecule comprises a nucleotide sequence encoding a cleavage domain.

The composition, when administered to the subject in need thereof, can result in the generation of the synthetic antibody in the subject more quickly than the generation of an endogenous antibody in a subject who is administered an antigen to induce a humoral immune response. The composition can result in the generation of the synthetic antibody at least about 1 day, 2 days, 3 days, 4 days, 5 days, 6 days, 7 days, 8 days, 9 days, or 10 days before the generation of the endogenous antibody in the subject who was administered an antigen to induce a humoral immune response.

The recombinant nucleic acid sequence can be a heterologous nucleic acid sequence. The recombinant nucleic acid sequence can include one or more heterologous nucleic acid sequences.

The recombinant nucleic acid sequence can be an optimized nucleic acid sequence. Such optimization can increase or alter the immunogenicity of the antibody. Optimization can also improve transcription and/or translation. Optimization can include one or more of the following: low GC content leader sequence to increase transcription; mRNA stability and codon optimization; addition of a kozak sequence (e.g., GCC ACC) for increased translation; addition of an immunoglobulin (Ig) leader sequence encoding a signal peptide; addition of an internal IRES sequence and eliminating to the extent possible cis-acting sequence motifs (i.e., internal TATA boxes).

The recombinant nucleic acid sequence construct can include a heterologous nucleic acid sequence that encodes a heavy chain polypeptide, a fragment thereof, a variant thereof, or a combination thereof. The recombinant nucleic acid sequence construct can include a heterologous nucleic acid sequence that encodes a light chain polypeptide, a fragment thereof, a variant thereof, or a combination thereof. The recombinant nucleic acid sequence construct can also include a heterologous nucleic acid sequence that encodes a protease or peptidase cleavage site. The recombinant nucleic acid sequence construct can also include a heterologous nucleic acid sequence that encodes an internal ribosome entry site (IRES). An IRES may be either a viral IRES or a eukaryotic IRES. The recombinant nucleic acid sequence construct can include one or more leader sequences, in which each leader sequence encodes a signal peptide. The recombinant nucleic acid sequence construct can include one or more promoters, one or more introns, one or more transcription termination regions, one or more initiation codons, one or more termination or stop codons, and/or one or more polyadenylation signals. The recombinant nucleic acid sequence construct can also include one or more linker or tag sequences. The tag sequence can encode a hemagglutinin (HA) tag.

Upon expression, for example, but not limited to, in a cell, organism, or mammal, the heavy chain polypeptide and the light chain polypeptide can assemble into the synthetic antibody. In particular, the heavy chain polypeptide and the light chain polypeptide can interact with one another such that assembly results in the synthetic antibody being capable of binding the antigen. In other embodiments, the heavy chain polypeptide and the light chain polypeptide can interact with one another such that assembly results in the synthetic antibody being more immunogenic as compared to an antibody not assembled as described herein. In still other embodiments, the heavy chain polypeptide and the light chain polypeptide can interact with one another such that assembly results in the synthetic antibody being capable of eliciting or inducing an immune response against the antigen.

### 5. VECTORS

The recombinant nucleic acid sequence construct described above can be placed in one or more vectors. The one or more vectors can contain an origin of replication. The one or more vectors can be a plasmid, bacteriophage, bacterial artificial chromosome or yeast artificial chromosome. The one or more vectors can be either a self-replication extra chromosomal vector, or a vector which integrates into a host genome.

Vectors include, but are not limited to, plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. In some embodiments, the vector includes linear DNA, enzymatic DNA or synthetic DNA. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

The one or more vectors can be a heterologous expression construct, which is generally a plasmid that is used to introduce a specific gene into a target cell. Once the expression vector is inside the cell, the heavy chain polypeptide and/or light chain polypeptide that are encoded by the recombinant nucleic acid sequence construct is produced by the cellular-transcription and translation machinery ribosomal complexes. The one or more vectors can express large amounts of stable messenger RNA, and therefore proteins.

a. Expression Vector

The one or more vectors can be a circular plasmid or a linear nucleic acid. The circular plasmid and linear nucleic acid are capable of directing expression of a particular nucleotide sequence in an appropriate subject cell. The one or more vectors comprising the recombinant nucleic acid sequence construct may be chimeric, meaning that at least one of its components is heterologous with respect to at least one of its other components.

b. Plasmid

The one or more vectors can be a plasmid. The plasmid may be useful for transfecting cells with the recombinant nucleic acid sequence construct. The plasmid may be useful for introducing the recombinant nucleic acid sequence construct into the subject. The plasmid may also comprise a regulatory sequence, which may be well suited for gene expression in a cell into which the plasmid is administered.

The plasmid may also comprise a mammalian origin of replication in order to maintain the plasmid extrachromosomally and produce multiple copies of the plasmid in a cell. The plasmid may be pVAX, pCEP4 or pREP4 from Invitrogen (San Diego, Calif.), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may be pSE420 (Invitrogen, San Diego, Calif.), which may be used for protein production in Escherichia coli (E. coli). The plasmid may also be p YES2 (Invitrogen, San Diego, Calif.), which may be used for protein production in Saccharomyces cerevisiae strains of yeast. The plasmid may also be of the MAXBAC™ complete baculovirus expression system (Invitrogen, San Diego, Calif.), which may be used for protein production in insect cells. The plasmid may also be pcDNAI or pcDNA3 (Invitrogen, San Diego, Calif.), which may be used for protein production in mammalian cells such as Chinese hamster ovary (CHO) cells.

c. RNA

In one embodiment, the nucleic acid is an RNA molecule. In one embodiment, the RNA molecule is transcribed from a DNA sequence described herein. For example, in some embodiments, the RNA molecule is encoded by a DNA sequence at least 90% homologous to a DNA sequence encoding one of SEQ ID NOs: 1, 3, 5, 7, or a variant thereof or a fragment thereof. Accordingly, in one embodiment, the invention provides an RNA molecule encoding one or more of the MAbs or DMAbs. The RNA may be plus-stranded. Accordingly, in some embodiments, the RNA molecule can be translated by cells without needing any intervening replication steps such as reverse transcription. A RNA molecule useful with the invention may have a 5′ cap (e.g. a 7-methylguanosine). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. A RNA molecule useful with the invention may be single-stranded. A RNA molecule useful with the invention may comprise synthetic RNA. In some embodiments, the RNA molecule is a naked RNA molecule. In one embodiment, the RNA molecule is comprised within a vector.

In one embodiment, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the RNA.

In one embodiment, the RNA has both a cap on the 5′ end and a 3′ poly(A) tail which determine ribosome binding, initiation of translation and stability of RNA in the cell.

In one embodiment, the RNA is a nucleoside-modified RNA. Nucleoside-modified RNA have particular advantages over non-modified RNA, including for example, increased stability, low or absent innate immunogenicity, and enhanced translation.

d. Circular and Linear Vector

The one or more vectors may be one or more circular plasmids, which may transform a target cell by integration into the cellular genome or exist extrachromosomally (e.g., autonomous replicating plasmid with an origin of replication). The vector can be pVAX, pcDNA3.0, or provax, or any other expression vector capable of expressing the heavy chain polypeptide and/or light chain polypeptide encoded by the recombinant nucleic acid sequence construct.

Also provided herein is a linear nucleic acid, or linear expression cassette (“LEC”), that is capable of being efficiently delivered to a subject via electroporation and expressing the heavy chain polypeptide and/or light chain polypeptide encoded by the recombinant nucleic acid sequence construct. The LEC may be any linear DNA devoid of any phosphate backbone. The LEC may not contain any antibiotic resistance genes and/or a phosphate backbone. The LEC may not contain other nucleic acid sequences unrelated to the desired gene expression.

The LEC may be derived from any plasmid capable of being linearized. The plasmid may be capable of expressing the heavy chain polypeptide and/or light chain polypeptide encoded by the recombinant nucleic acid sequence construct. The plasmid can be pNP (Puerto Rico/34) or pM2 (New Caledonia/99). The plasmid may be WLV009, pVAX, pcDNA3.0, or provax, or any other expression vector capable of expressing the heavy chain polypeptide and/or light chain polypeptide encoded by the recombinant nucleic acid sequence construct.

The LEC can be perM2. The LEC can be perNP. perNP and perMR can be derived from pNP (Puerto Rico/34) and pM2 (New Caledonia/99), respectively.

e. Viral Vectors

In one embodiment, viral vectors are provided herein which are capable of delivering a nucleic acid of the invention to a cell. The expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector comprises an origin of replication functional in at least one organism, a promoter sequence, convenient restriction endonuclease sites, and one or more selectable markers. (See, e.g., WO 01/96584; WO 01/29058; and U.S. Pat. No. 6,326,193). Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

f. Method of Preparing the Vector

Provided herein is a method for preparing the one or more vectors in which the recombinant nucleic acid sequence construct has been placed. After the final subcloning step, the vector can be used to inoculate a cell culture in a large scale fermentation tank, using known methods in the art.

In other embodiments, after the final subcloning step, the vector can be used with one or more electroporation (EP) devices. The EP devices are described herein in more herein.

The one or more vectors can be formulated or manufactured using a combination of known devices and techniques, and may be manufactured using a plasmid manufacturing technique that is described in U.S. provisional application U.S. Ser. No. 60/939,792, which was filed on May 23, 2007. In some examples, the DNA plasmids described herein can be formulated at concentrations greater than or equal to 10 mg/mL. The manufacturing techniques also include or incorporate various devices and protocols that are commonly known to those of ordinary skill in the art, in addition to those described in U.S. Ser. No. 60/939,792, including those described in U.S. Pat. No. 7,238,522, which issued on Jul. 3, 2007. The above-referenced application and patent, U.S. Ser. No. 60/939,792 and U.S. Pat. No. 7,238,522, respectively, are hereby incorporated in their entirety.

### 6. METHOD OF GENERATING THE SYNTHETIC ANTIBODY

The present invention also relates a method of generating the synthetic antibody. The method can include administering the composition to the subject in need thereof by using the method of delivery described in more detail herein. Accordingly, the synthetic antibody is generated in the subject or in vivo upon administration of the composition to the subject.

The method can also include introducing the composition into one or more cells, and therefore, the synthetic antibody can be generated or produced in the one or more cells. The method can further include introducing the composition into one or more tissues, for example, but not limited to, skin and muscle, and therefore, the synthetic antibody can be generated or produced in the one or more tissues.

### 7. EXCIPIENTS AND OTHER COMPONENTS OF THE COMPOSITION

The composition may further comprise a pharmaceutically acceptable excipient. The pharmaceutically acceptable excipient can be functional molecules such as vehicles, adjuvants, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the composition. The composition may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the composition is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient can be an adjuvant in addition to the checkpoint inhibitor antibodies of the invention. The additional adjuvant can be other genes that are expressed in an alternative plasmid or are delivered as proteins in combination with the plasmid above in the composition. The adjuvant may be selected from the group consisting of: α-interferon(IFN-α), (3-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MEW, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, PD-1, IL-10, IL-12, IL-18, or a combination thereof.

Other genes that can be useful as adjuvants in addition to the antibodies of the invention include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, IL-22, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The composition may further comprise a genetic facilitator agent as described in U.S. Ser. No. 021,579 filed Apr. 1, 1994, which is fully incorporated by reference.

The composition may comprise DNA at quantities of from about 1 nanogram to 100 milligrams; about 1 microgram to about 10 milligrams; or preferably about 0.1 microgram to about 10 milligrams; or more preferably about 1 milligram to about 2 milligram. In some preferred embodiments, composition according to the present invention comprises about 5 nanogram to about 1000 micrograms of DNA. In some preferred embodiments, composition can contain about 10 nanograms to about 800 micrograms of DNA. In some preferred embodiments, the composition can contain about 0.1 to about 500 micrograms of DNA. In some preferred embodiments, the composition can contain about 1 to about 350 micrograms of DNA. In some preferred embodiments, the composition can contain about 25 to about 250 micrograms, from about 100 to about 200 microgram, from about 1 nanogram to 100 milligrams; from about 1 microgram to about 10 milligrams; from about 0.1 microgram to about 10 milligrams; from about 1 milligram to about 2 milligram, from about 5 nanogram to about 1000 micrograms, from about 10 nanograms to about 800 micrograms, from about 0.1 to about 500 micrograms, from about 1 to about 350 micrograms, from about 25 to about 250 micrograms, from about 100 to about 200 microgram of DNA.

The composition can be formulated according to the mode of administration to be used. An injectable pharmaceutical composition can be sterile, pyrogen free and particulate free. An isotonic formulation or solution can be used. Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polycations or polyanions.

### 8. METHOD OF IN VIVO POST-TRANSLATIONAL MODIFICATION

The present invention is also directed to a method post-translationally modifying a synthetic protein in a subject. Post-translationally modifying a synthetic protein in a subject can be used to treat and/or prevent disease in the subject by providing a biologically active protein. The method can include administering the herein disclosed composition to the subject. The subject administered the composition can have an increased or boosted protein activity as compared to a subject administered the without post-translational modification. In some embodiments, the protein activity can be increased by about 0.5-fold to about 15-fold, about 0.5-fold to about 10-fold, or about 0.5-fold to about 8-fold. Alternatively, the protein activity in the subject administered the composition can be increased by at least about 0.5-fold, at least about 1.0-fold, at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, or at least about 15.0-fold.

In still other alternative embodiments, the protein activity in the subject administered the composition can be increased about 50% to about 1500%, about 50% to about 1000%, or about 50% to about 800%. In other embodiments, the protein activity in the subject administered the composition can be increased by at least about 50%, at least about 100%, at least about 150%, at least about 200%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1000%, at least about 1050%, at least about 1100%, at least about 1150%, at least about 1200%, at least about 1250%, at least about 1300%, at least about 1350%, at least about 1450%, or at least about 1500%.

The dose can be between 1 μg to 10 mg active component/kg body weight/time, and can be 20 μg to 10 mg component/kg body weight/time. The composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of doses for effective treatment can be 1, 2, 3, 4, 5, 6, 7, 8, 9, or 10.

### 9. METHOD OF DELIVERY OF THE COMPOSITION

The present invention also relates to a method of delivering the composition to the subject in need thereof. The method of delivery can include, administering the composition to the subject. Administration can include, but is not limited to, DNA injection with and without in vivo electroporation, liposome mediated delivery, and nanoparticle facilitated delivery.

The mammal receiving delivery of the composition may be human, primate, non-human primate, cow, cattle, sheep, goat, antelope, bison, water buffalo, bison, bovids, deer, hedgehogs, elephants, llama, alpaca, mice, rats, and chicken.

The composition may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal, intranasal, intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The composition may be administered by traditional syringes, needleless injection devices, “microprojectile bombardment gone guns”, or other physical methods such as electroporation (“EP”), “hydrodynamic method”, or ultrasound.

a. Electroporation

Administration of the composition via electroporation may be accomplished using electroporation devices that can be configured to deliver to a desired tissue of a mammal, a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electroporation may be accomplished using an in vivo electroporation device, for example CELLECTRA EP system (Inovio Pharmaceuticals, Plymouth Meeting, Pa.) or Elgen electroporator (Inovio Pharmaceuticals, Plymouth Meeting, Pa.) to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 μs, 20 μs, 10 μs or 1 μs, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the composition of the present invention, include those described in U.S. Pat. No. 7,245,963 by Draghia-Akli, et al., U.S. Patent Pub. 2005/0052630 submitted by Smith, et al., the contents of which are hereby incorporated by reference in their entirety. Other electroporation devices and electroporation methods that may be used for facilitating delivery of the composition include those provided in co-pending and co-owned U.S. patent application Ser. No. 11/874,072, filed Oct. 17, 2007, which claims the benefit under 35 USC 119(e) to U.S. Provisional Applications Ser. No. 60/852,149, filed Oct. 17, 2006, and 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference.

U.S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference.

The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes The electrodes described in U.S. Pat. No. 7,245,963 and U.S. Patent Pub. 2005/005263 are preferably 20 mm long and 21 gauge.

Additionally, contemplated in some embodiments, that incorporate electroporation devices and uses thereof, there are electroporation devices that are those described in the following patents: U.S. Pat. No. 5,273,525 issued Dec. 28, 1993, U.S. Pat. No. 6,110,161 issued Aug. 29, 2000, U.S. Pat. No. 6,261,281 issued Jul. 17, 2001, and U.S. Pat. No. 6,958,060 issued Oct. 25, 2005, and U.S. Pat. No. 6,939,862 issued Sep. 6, 2005. Furthermore, patents covering subject matter provided in U.S. Pat. No. 6,697,669 issued Feb. 24, 2004, which concerns delivery of DNA using any of a variety of devices, and U.S. Pat. No. 7,328,064 issued Feb. 5, 2008, drawn to method of injecting DNA are contemplated herein. The above-patents are incorporated by reference in their entirety.

### 10. METHOD OF TREATMENT

Also provided herein is a method of treating, protecting against, and/or preventing a disease, disorder or infection in a subject in need thereof by administering one or more compositions described herein. In one embodiment, the methods comprise administering one or more synthetic protein constructs such that a synthetic protein is generated in the subject. In one embodiment, the methods comprise administering one or more genetic constructs and proteins such that secreted proteins, or synthetic antigens, will be recognized as foreign by the immune system, which will mount an immune response that can include antibodies made against the one or more antigens. In one embodiment, the methods comprise administering one or more DMAb constructs. In one embodiment, the methods comprise administering one or more modifier protein constructs.

In one embodiment, administering a nucleic acid encoding a synthetic protein and a nucleic acid encoding a modifier protein provides a biologically active, post-translationally modified synthetic protein. The method can include administering the composition to the subject. Administration of the composition to the subject can be done using the method of delivery described above.

In one aspect, the invention provides a method of treating, protecting against, and/or preventing a disease or disorder, wherein the synthetic protein treats the disease or disorder. In certain embodiments, the invention provides a method of treating, protecting against, and/or preventing a HIV Virus infection. In one embodiment, the method treats, protects against, and/or prevents a disease associated with HIV. In one embodiment, the method of treating or preventing HIV comprises administering the nucleic acid encoding a synthetic eCD4-Ig protein and the nucleic acid encoding TPST2 as described elsewhere herein.

Upon generation of the synthetic protein and the modifier protein in the subject, the modifier antibody can post-translationally modify the synthetic protein. Such modification can activate the enzymatic, binding, or antigen presenting activity of the synthetic protein, thereby treating, protecting against, and/or preventing the disease in the subject.

The composition dose can be between 1 μg to 10 mg active component/kg body weight/time, and can be 20 μg to 10 mg component/kg body weight/time. The composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of composition doses for effective treatment can be 1, 2, 3, 4, 5, 6, 7, 8, 9, or 10.

The composition can comprise 1 or more, 2 or more, 3 or more, 4 or more, 5 or more, 6 or more, 7 or more, 8 or more, 9 or more, or 10 or more nucleic acids encoding proteins. The composition may comprise 1 or more, 2 or more, 3 or more, 4 or more, 5 or more, 6 or more, 7 or more, 8 or more, 9 or more, or 10 or more nucleic acids encoding modifier proteins.

The nucleic acid encoding the synthetic protein and the nucleic acid encoding the modifier protein may be administered at the same time or at different times. In one embodiment, the nucleic acid encoding the synthetic protein and the nucleic acid encoding the modifier protein are administered simultaneously. In one embodiment, the nucleic acid encoding the synthetic protein is administered before the nucleic acid encoding the modifier protein. In one embodiment, the nucleic acid encoding the modifier protein is administered before the nucleic acid encoding the synthetic protein.

In certain embodiments, the nucleic acid encoding the synthetic protein is administered 1 or more days, 2 or more days, 3 or more days, 4 or more days, 5 or more days, 6 or more days, 7 or more days, 8 or more days, 9 or more days, 10 or more days, 11 or more days, 12 or more days, 13 or more days, or 14 or more days after the nucleic acid encoding the modifier protein is administered. In certain embodiments, the Nucleic acid encoding the synthetic protein is administered 1 or more weeks, 2 or more weeks, 3 or more weeks, 4 or more weeks, 5 or more weeks, 6 or more weeks, 7 or more weeks, 8 or more weeks, 9 or more weeks, or 10 or more weeks after the Nucleic acid encoding the modifier protein is administered. In certain embodiments, the Nucleic acid encoding the synthetic protein is administered 1 or more months, 2 or more months, 3 or more months, 4 or more months, 5 or more months, 6 or more months, 7 or more months, 8 or more months, 9 or more months, 10 or more months, 11 or more months, or 12 or more months after the Nucleic acid encoding the modifier protein is administered.

In certain embodiments, the Nucleic acid encoding the modifier protein is administered 1 or more days, 2 or more days, 3 or more days, 4 or more days, 5 or more days, 6 or more days, 7 or more days, 8 or more days, 9 or more days, 10 or more days, 11 or more days, 12 or more days, 13 or more days, or 14 or more days after the Nucleic acid encoding the synthetic protein is administered. In certain embodiments, the Nucleic acid encoding the modifier protein is administered 1 or more weeks, 2 or more weeks, 3 or more weeks, 4 or more weeks, 5 or more weeks, 6 or more weeks, 7 or more weeks, 8 or more weeks, 9 or more weeks, or 10 or more weeks after the Nucleic acid encoding the synthetic protein is administered. In certain embodiments, the Nucleic acid encoding the modifier protein is administered 1 or more months, 2 or more months, 3 or more months, 4 or more months, 5 or more months, 6 or more months, 7 or more months, 8 or more months, 9 or more months, 10 or more months, 11 or more months, or 12 or more months after the Nucleic acid encoding the synthetic protein is administered.

In certain embodiments, the Nucleic acid encoding the modifier protein and Nucleic acid encoding the synthetic protein are administered once. In certain embodiments, the Nucleic acid encoding the modifier protein and/or the Nucleic acid encoding the synthetic protein are administered more than once. In certain embodiments, administration of the Nucleic acid encoding the modifier protein and nucleic acid encoding the synthetic protein provides immediate, persistent, and systemic immune responses.

The composition dose can be between 1 μg to 10 mg active component/kg body weight/time, and can be 20 μg to 10 mg component/kg body weight/time. The composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of composition doses for effective treatment can be 1, 2, 3, 4, 5, 6, 7, 8, 9, or 10.

### 11. EXAMPLES

### Example 1: Synthetic DNA Delivery by Electroporation Promotes Robust In Vivo Sulfation of Broadly Neutralizing Anti-HIV Immunoadhesin ECD4-Ig

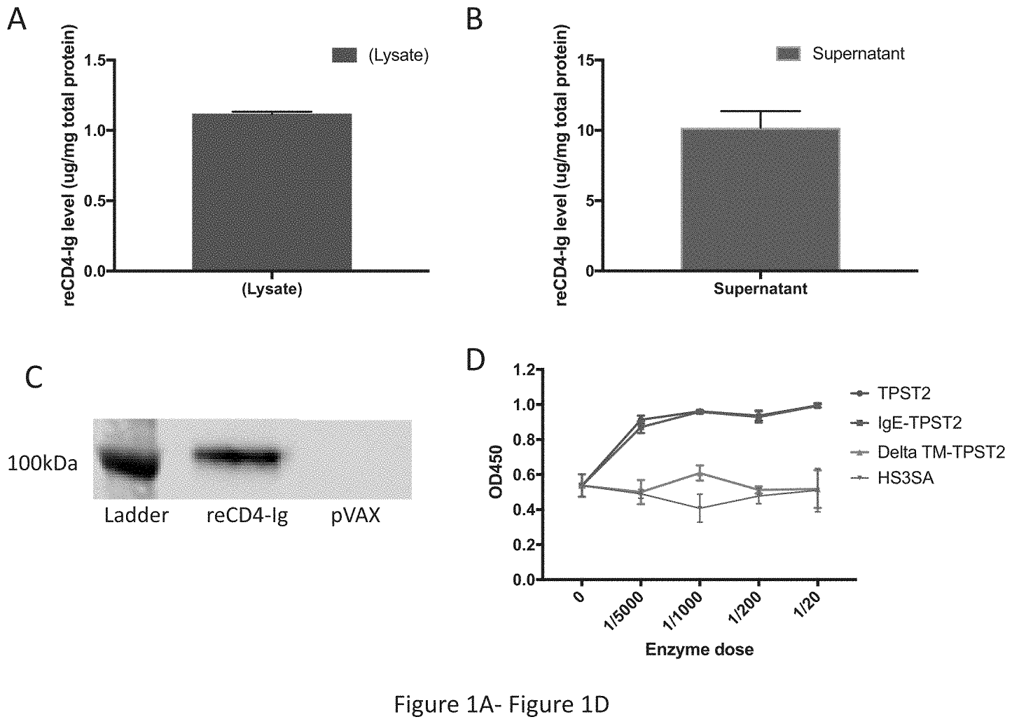

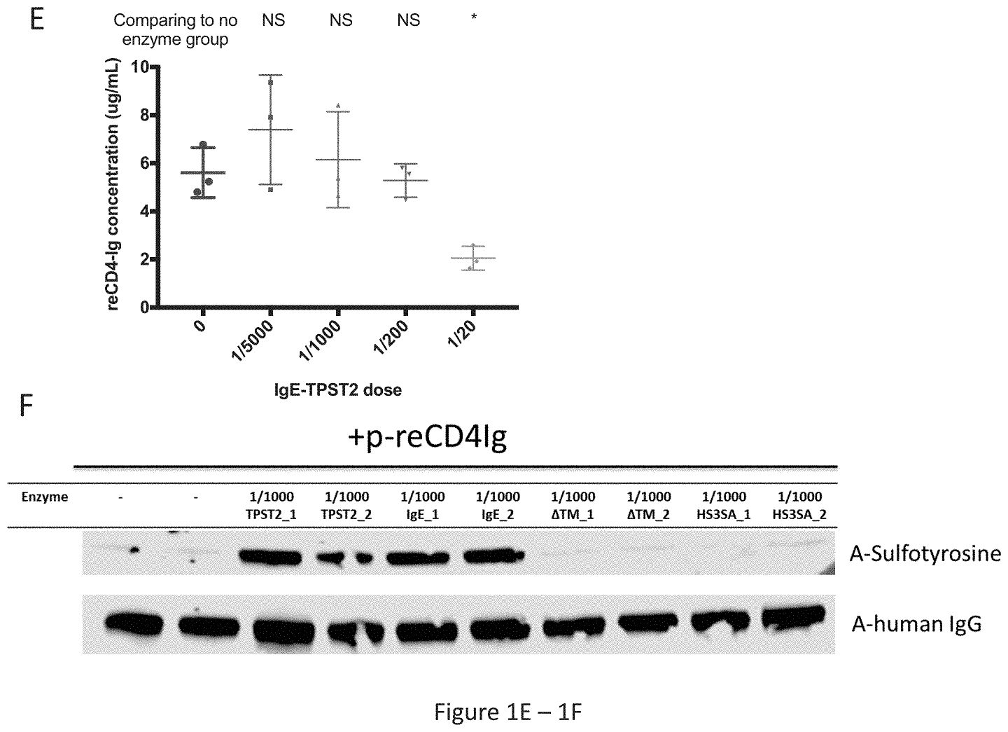

Transfection of HEK293T cells enables expression and secretion of ReCD4-Ig in vitro A transgene encoding ReCD4-Ig with an N-terminal IgG kappa-leader sequence was designed and then synthesized de novo and cloned into the pGX00001backbone plasmid. The transgene nucleotide sequence was optimized for codon biases in both mouse and human and mRNA transcript structure and stability (Graf et al., 2004; Patel et al., 2017). The N-terminal IgG leader sequence is incorporated to facilitate targeting of the transgene to endoplasmic reticulum and will promote secretion (Haryadi et al., 2015). Robust expression of ReCD4-Ig was observed in cell lysates and supernatant (FIG. 1A-B). Western blot of transfection supernatant with anti-human IgG confirms secretion of ReCD4-Ig by the transfected cells (FIG. 1C).

Co-Transfection of HEK293T Cells with DNA-Encoded ReCD4-Ig and TPST2 Variants Allows In Vitro Sulfation of ReCD4-Ig

Tyrosine sulfation is a specific post translational modification catalyzed by a unique collection of specific tyrosylprotein sulfotransferases (TPST) enzymes. In humans there exist two different TPST isoforms (TPST1 & TPST2). While their function is not completely understood, these enzymes are implicated in several altering important protein biological activates including modifying protein half-life, protein processing, and modifying protein-protein interactions. Of relevance, sulfation of CCR5 plays an important role in HIV binding and entry modification at the cell surface. To determine whether ReCD4-Ig can be induced to become sulfated which is important for biological folding, binding of eCD4-Ig and activity for gp120, HEK293T cells were co-transfected with plasmid-encoding ReCD4-Ig (p-ReCD4-Ig) and 4 different human enzyme constructs encoding p-TPST2, p-IgE-TPST2, p-ATM-TPST2, and p-HS3 SA. As ReCD4-Ig is targeted to the secretory pathway early in the translation process by the IgG leader sequence, tyrosine sulfation of ReCD4-Ig should only occur if the TPST2 variant is expressed in at least one of the cellular secretory compartments. To highlight the ability to target TPST2 to the right subcellular compartment, HEK293T cells were co-transfected with p-ReCD4-Ig and plasmids encoding TPST2 enzyme variants. The p-IgE-TPST2, a construct with an IgE leader sequence incorporated upstream to TPST2, was predicted to sulfate ReCD4-Ig since the IgE leader sequence facilitates trafficking of TPST2 into the endoplasmic reticulum during translation. A second TPST2 construct with a deletion in the transmembrane (TM) motif, p-ATM-TPST2, was not expected to sulfate ReCD4-Ig since the TM deletion removes the signal anchor sequence required for targeting of TPST2 to the secretory compartment. Finally, a control plasmid, p-HS3 SA, was tested. HS3 SA is a Golgi-resident enzyme which can transfer sulfate groups to heparin sulfate and has a similar catalytic site as compared to TPST2 (Teramoto et al., 2013). Varying doses of DNA-encoded enzymes (1:5000 to 1:20, enzyme: ReCD4-Ig) were used to determine the minimal dose of enzyme required to maximize sulfation of ReCD4-Ig. Using an anti-sulfotyrosine binding ELISA on cell supernatant, higher sulfation was observed for both TPST2 and IgE-TPST2 groups, even at the lowest enzyme dose of 1:5000, as compared to the baseline ReCD4-Ig only group (FIG. 1D). Furthermore, for both TPST2 and IgE-TPST2, sulfation signals were saturated at a remarkable 1:1000 enzyme dose, and higher dose of DNA-encoded enzyme did not contribute to increased sulfation. In comparison, consistent with the hypothesis, RecCD4-Ig sulfation for both the ATM-TPST2 and HS3 SA groups were not higher than the baseline, even at the highest 1:20 enzyme dose. The lack of sulfation with the HS3 SA group indicates remarkable specificity of sulfotransferases. To further confirm enzyme-mediated sulfation of ReCD4-Ig, the supernatants were analyzed with Western blots, where anti-human IgG bands in the lower panel serve as the loading controls (FIG. 1F). Again, stronger sulfotyrosine bands were observed for the 1:1000 TPST2 and IgE-TPST2 groups than for the ReCD4-Ig only, ATM, and HS3 SA groups. Taken together, these results suggest that the DNA-encoded TPST2, and IgE-TPST2 can mediate in vitro sulfation of ReCD4-Ig at a remarkably low dose.

Incorporation of the N-Terminal IgE Leader Sequence Enhances Targeting of TPST2 to TGN

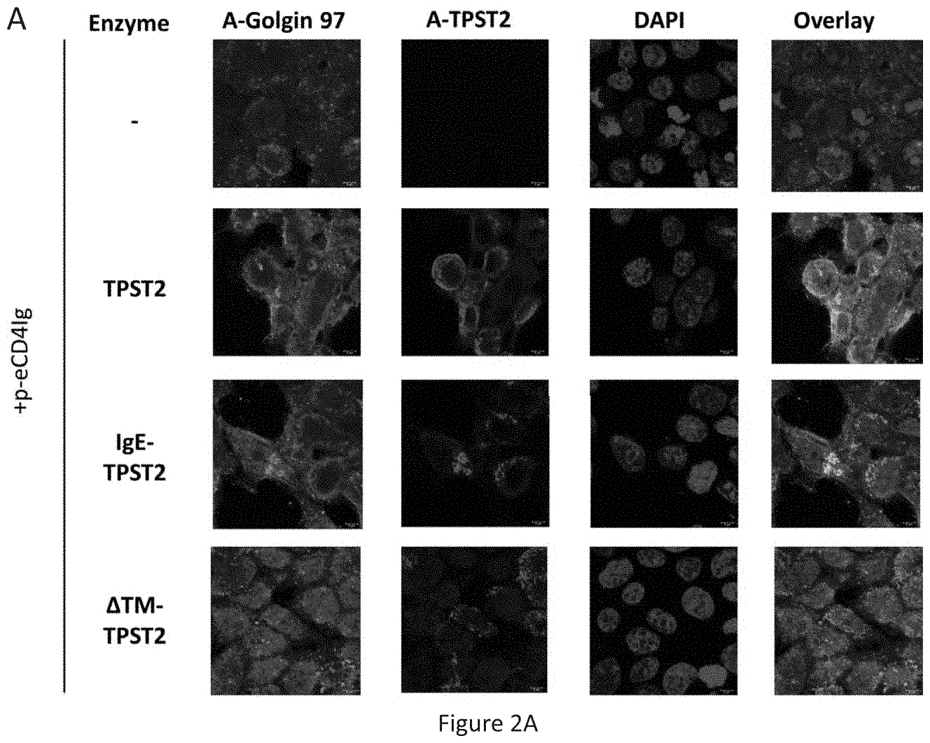

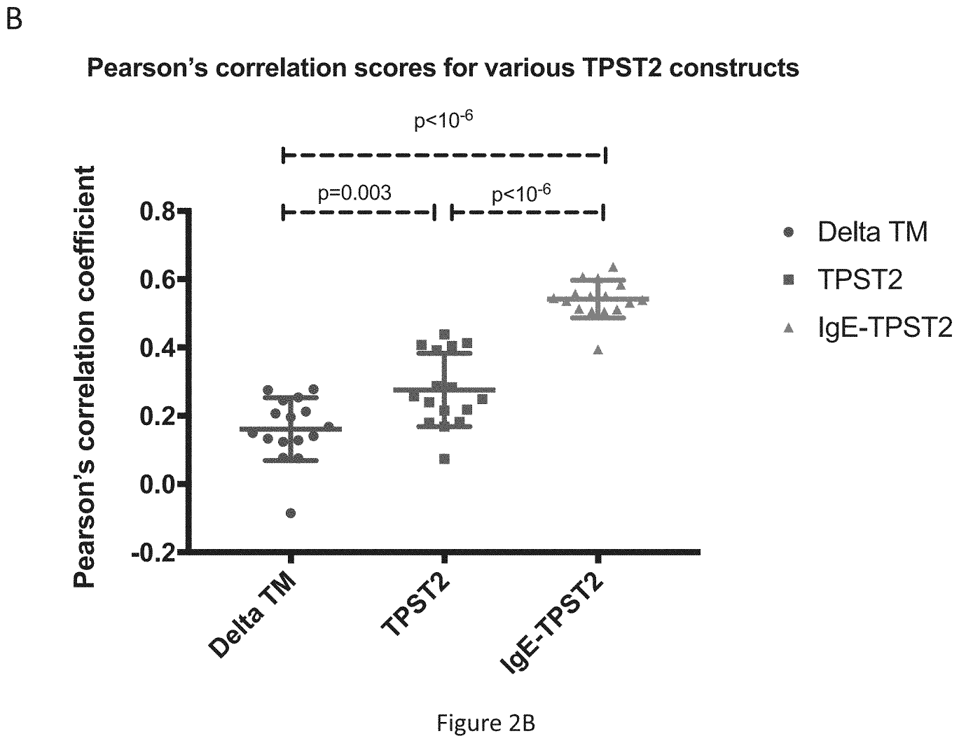

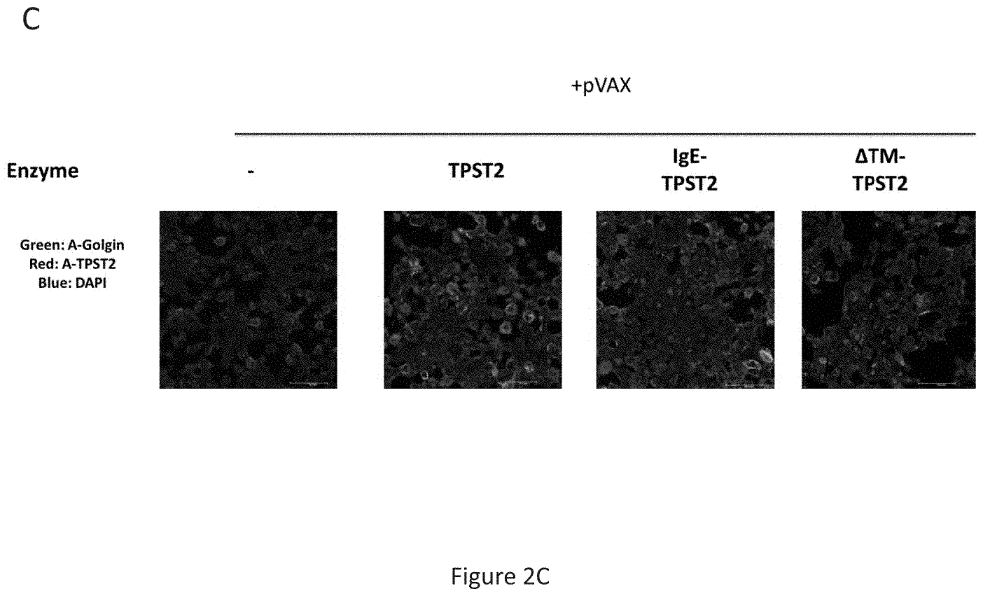

Fluorescence microscopy was used to determine whether DNA-encoded enzymes can traffic to cellular secretory compartments and if the IgE sequence would improve targeting. HEK293T cells were transfected with either ReCD4-Ig only or ReCD4-Ig in combination with TPST2, IgE-TPST2, or ATM-TPST2. 48 hours after transfections, cells were harvested and stained with DAPI (blue), anti-TPST2 (red), and anti-Golgin 97 (green). Confocal microscopy images of harvested cells illustrate robust expression of TPST2, IgE-TPST2 and ATM-TPST2 upon transfection (FIG. 2A). More importantly, IgE-TPST2 appears to localize with Golgin 97 to a greater extent than TPST2, whereas ATM-TPST2 does not localize with Golgin 97. To quantify the extent of colocalization between Golgin 97 and TPST2 variants, the pearson correlation coefficients between red and green channels were analyzed for 16 regions of interests for each group (FIG. 2B). The mean pearson coefficients of ATM-TPST2, TPST2 and IgE-TPST2 are 0.161, 0.275 and 0.542 respectively. The computed F-statistics from one-way ANOVA analysis is 79.67, which corresponds to p-value<0.0001 with 2 and 45 treatment and residual degrees freedom, respectively. Post-hoc pairwise T-test with Holm adjustment shows pearson coefficient for the IgE-TPST2 group is significantly higher than that for the TPST2 group (p<10′). Further, HEK293T cells were transfected with only enzymes and determined the localization patterns of TPST2, IgE-TPST2 and ATM-TPST2 with Golgin 97 are similar to those when the cells are co-transfected with ReCD4-Ig (FIG. 2C)). Taken together, these results suggest that while both TPST2 and IgE-TPST2 can traffic to TGN, IgE-TPST2 can be targeted to the secretory compartment more efficiently than TPST2. This finding supports that the N-terminal IgE leader sequence is recognized by signal recognition particle (SRP) more efficiently than the internal signal anchorage sequence for TPST2 (which is also its transmembrane domain). The IgE-TPST2 construct was selected for further study in the in vivo experiments. The improved targeting of IgE-TPST2 to the secretory compartments, cytosolic expression of IgE-TPST2 and off-target effects were arguably reduced.

DNA/EP Allows In Vivo Expression of IgE-TPST2 and ReCD4-Ig

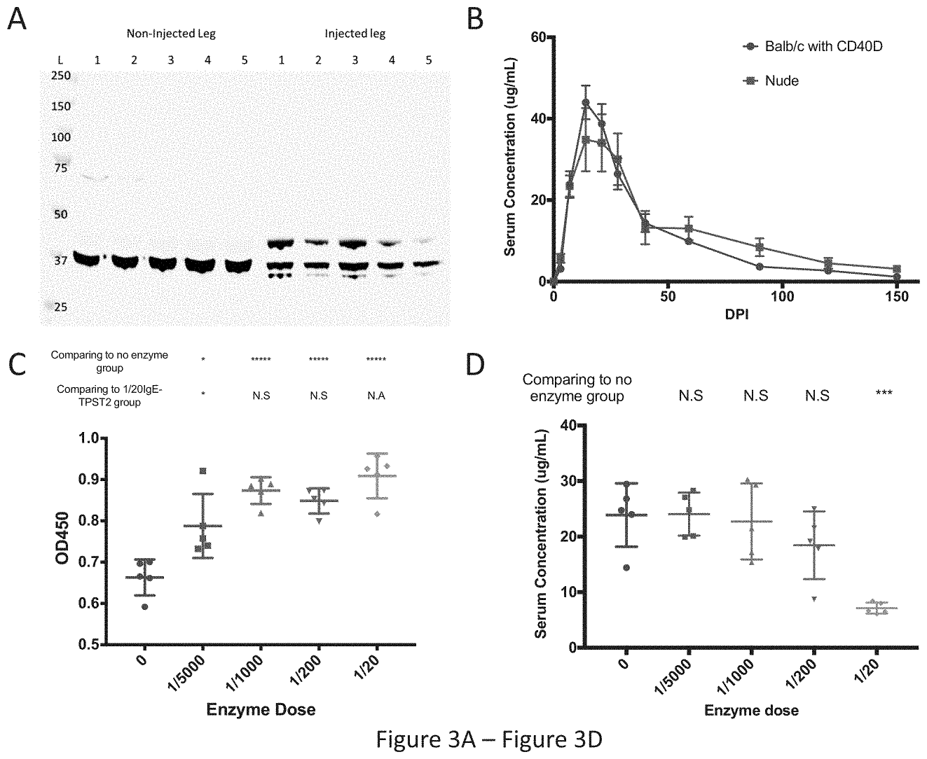

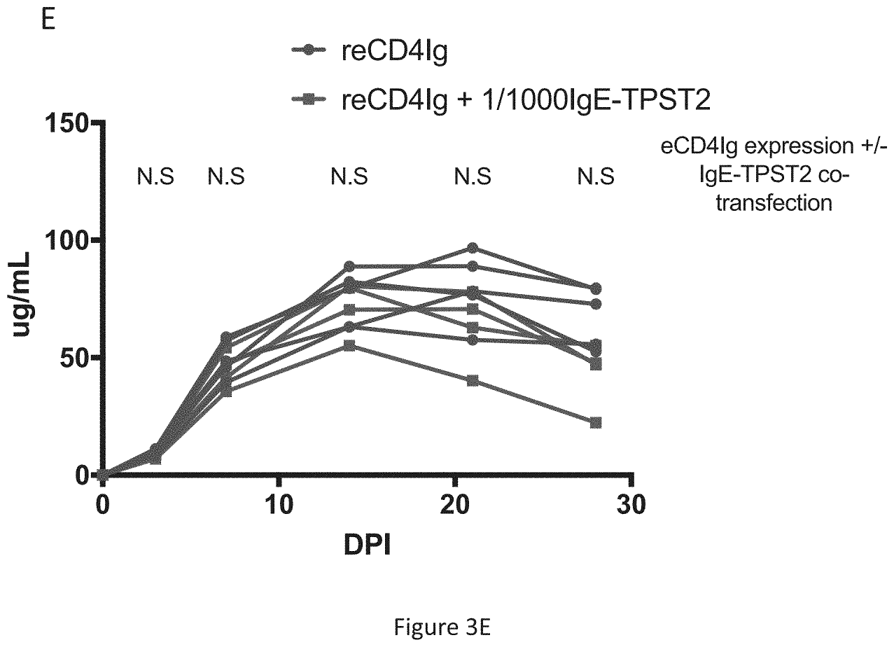

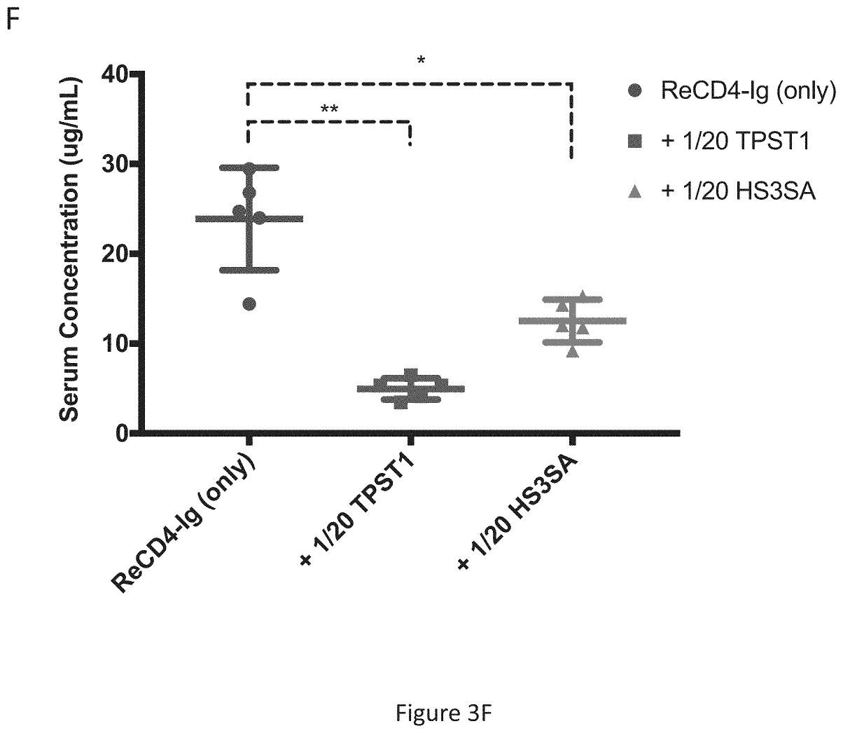

Next it was determined whether IgE-TPST2 and ReCD4-Ig can be expressed in vivo by intramuscular injection of DNA followed by electroporation. Transiently depleted balb/c mice were injected with DNA-encoded IgE-TPST2 into the tibialis anterior (TA) muscle, followed by intramuscular electroporation (IM-EP) using CELLECTRA® 3P device as previously described. One week after injection, mice were sacrificed and expression of IgE-TPST2 in the muscles was detected with Western blot. TA muscles in the contralateral legs were also analyzed for comparison. Strong expression of IgE-TPST2 (human form) in the injected muscles at around 43 kDa, and expression of endogenous mice TPST2 in the contralateral TAs at around 42 kDa was observed (FIG. 3A). The results demonstrate robustness of DNA/EP mediated delivery as IgE-TPST2 expression is consistently observed in every animal treated. To determine whether ReCD4-Ig can be redelivered, B6.Cg-Foxn1nu/J (nude) mice were injected with p-ReCD4-Ig Since ReCD4-Ig sequence is RhM based, strong anti-drug antibodies could develop in immune competent mice and influence the expression profile of ReCD4-Ig. Thus, immunodeficient B6.Cg-Foxn1nu/J (nude) were used to determine the initial in vivo expression of ReCD4-Ig. High level of expression of ReCD4-Ig was observed, which peaks at 35 ug/mL on Day 14 post injection (d.p.i) (FIG. 3B). Remarkably, a level of 5.7 ug/mL 3 d.p.i was detected and expression lasted for at least 150 days with a level of 3.1 ug/mL on the last time point. Similar expression profiles of ReCD4-Ig in balb/c mice were observed as compared to nude mice, especially at earlier timepoints (up until 42 d.p.i). Even though ReCD4-Ig expression in balb/c is slightly lower than that in nude mice at later time points, expression in balb/c remains detectable for 150 days, with

Low Dose of DNA-Encoded IgE-TPST2 can Allow In Vivo Sulfation of ReCD4-Ig

Next, the ability of IgE-TPST2 to sulfate ReCD4-Ig was tested in vivo. Balb/c mice were transiently depleted and given p-ReCD4-Ig co-formulated with varying doses of p-IgE-TPST2 intramuscularly, followed by IM-EP. The identical DNA doses of IgE-TPST2 as the in vitro experiments (1:5000 to 1:20 relative of ReCD4-Ig dose) were used to study a minimal level of enzyme required to optimize sulfation of ReCD4-Ig. A 1:1000 dose of IgE-TPST2 can saturate the sulfation OD450 signals detected as compared to 1:20 IgE-TPST2 group (FIG. 3c). Additionally, sulfation of ReCD4-Ig was significantly higher, even at a lower 1:5000 dose of the enzyme, as compared to the baseline ReCD4-Ig only group. Previous studies have reported that co-transfection of a high dose of TPST2 and its target proteins (eCD4-Ig or trypsinogen) in vitro lead to decreased secretion of the target proteins (Chen et al., 2016; Ronai et al., 2009). A similar phenomenon was observed both in the in vitro and in vivo experiments (FIG. 1E and FIG. 3D). A high dose (1:20) of IgE-TPST2 co-transfected with ReCD4-Ig resulted in 67% and 70% decreases in the expression of ReCD4-Ig in transfection supernatants and mice sera, respectively, as compared to ReCD4-Ig only groups. Additionally, suppression of ReCD4-Ig secretion is not directly driven by IgE-TPST2-mediated sulfation since co-administration of ReCD4-Ig and 1:20 DNA dose of HS3SA, an enzyme that cannot sulfate ReCD4-Ig (FIG. 1D), still results in a 52% reduction in ReCD4-Ig expression (FIG. 3F). However, at the minimal dose of 1:1000 required for optimal sulfation of ReCD4-Ig, a difference was not observed in ReCD4-Ig expression between ReCD4-Ig only and ReCD4-Ig+1:1000 IgE-TPST2 groups 7 d.p.i (FIG. 3D). To confirm ReCD4-Ig expression was not affected by co-transfection with IgE-TPST2 at low dose, sera expression of ReCD4-Ig in mice injected was followed with either pReCD4-Ig alone or pReCD4-Ig+1:1000 p-IgE-TPST2 over time (FIG. 3e). Again, similar ReCD4-Ig expression profile was observed in both groups. Taken together, these results illustrate that plasmid encoded enzymes delivered by electroporation can enable in vivo sulfation of ReCD4-Ig at a remarkably low dose, which does not affect expression profile of ReCD4-Ig.

In Vivo Sulfation Increases Potency of ReCD4-Ig

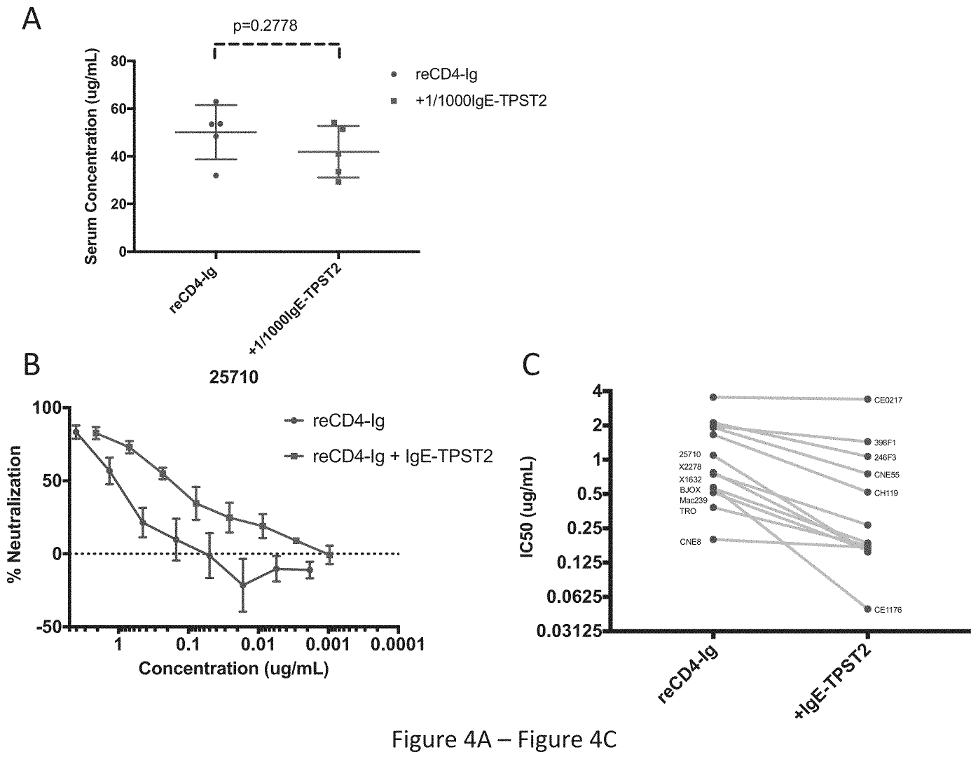

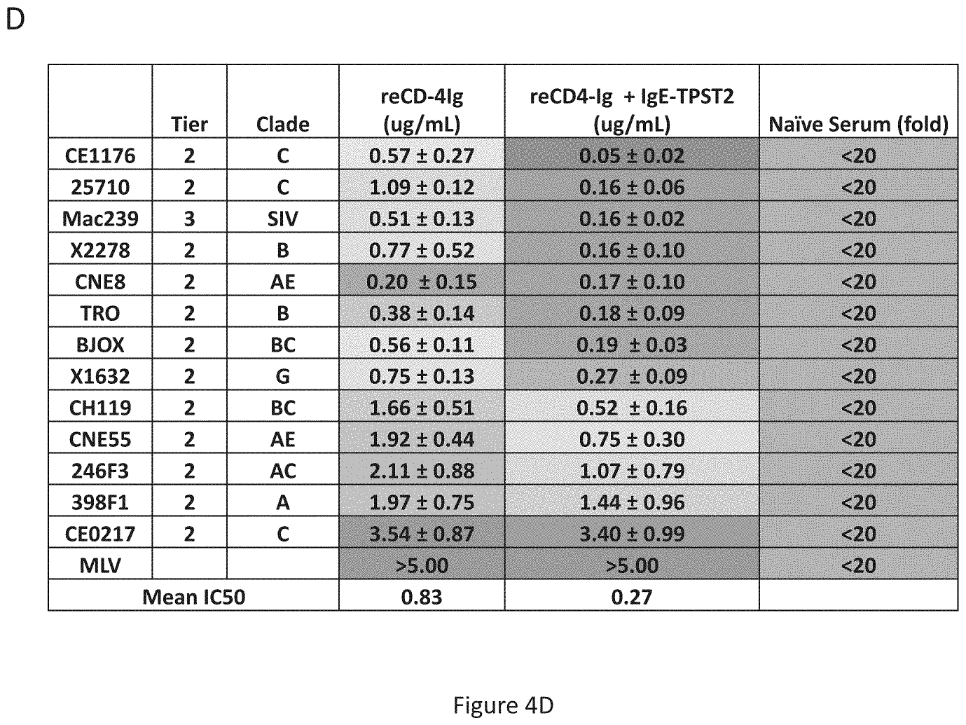

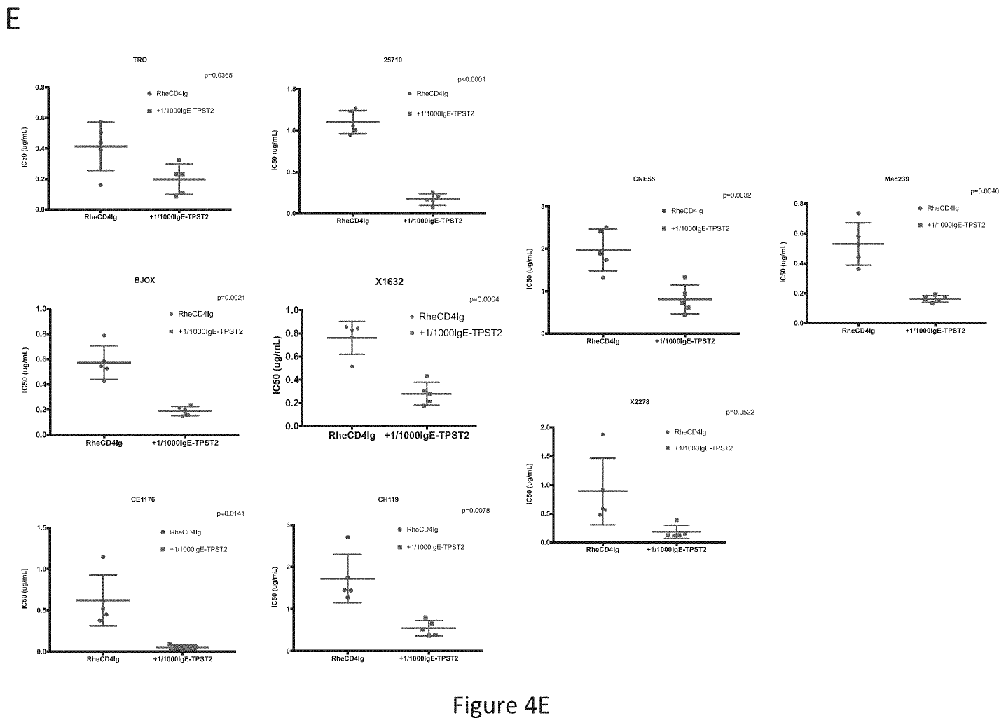

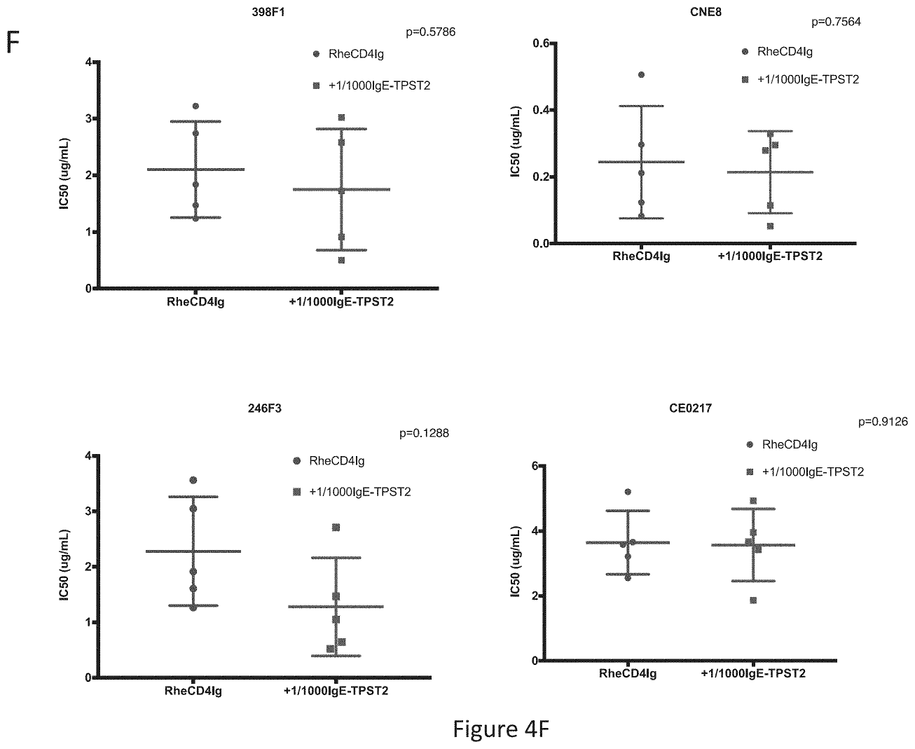

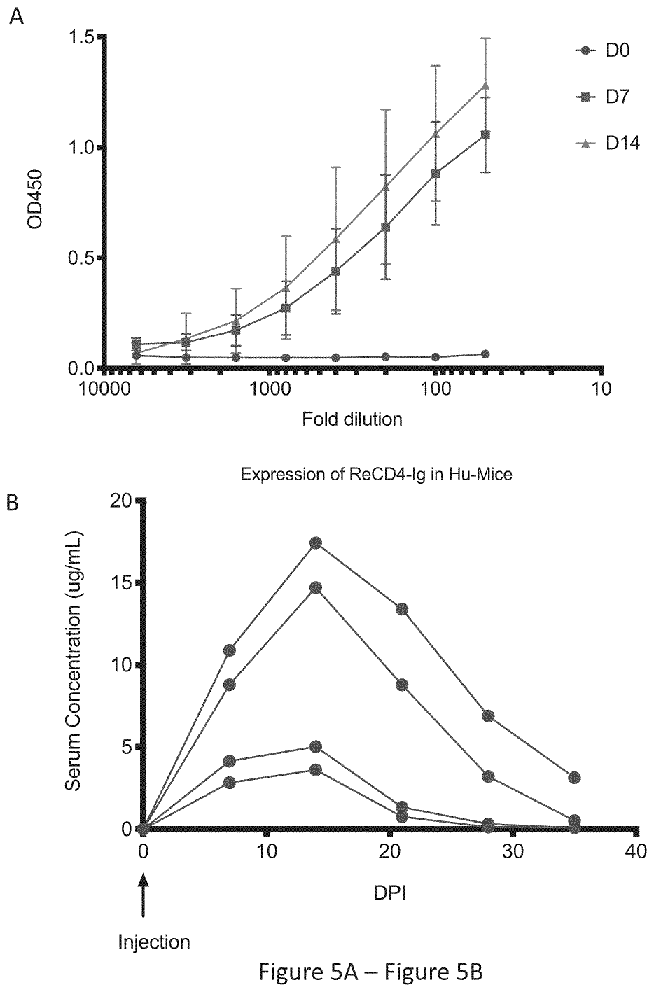

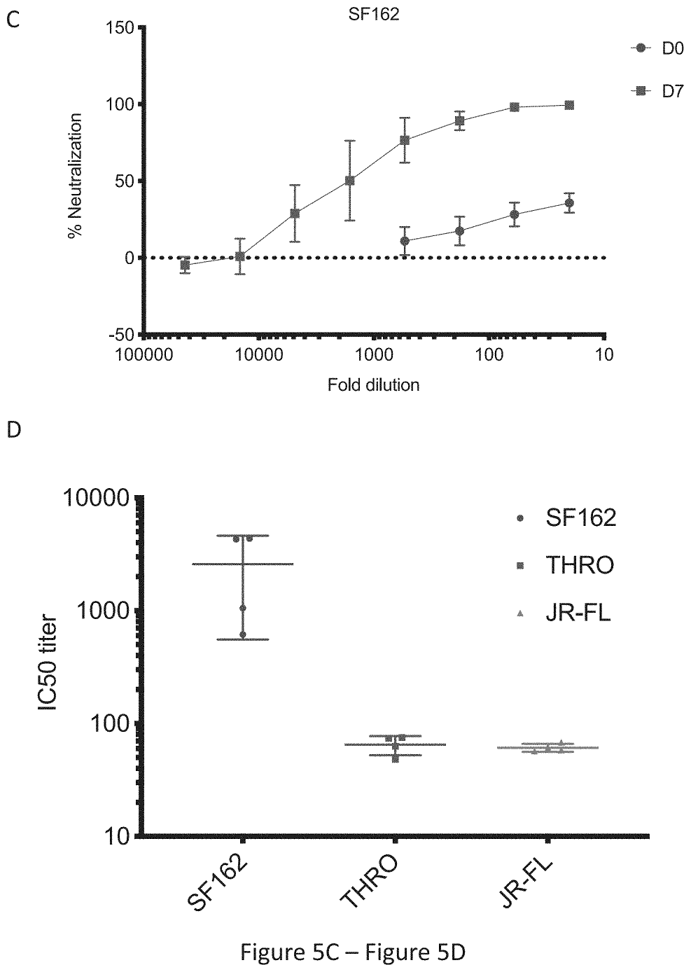

Next, it was determined if in vivo sulfation of ReCD4-Ig can enhance its potency by analyzing sera of injected mice in an ex vivo neutralization assay. To collect sufficient mouse sera, balb/c were injected with p-ReCD4-Ig alone or in combination with 1:1000 dose of p-IgE-TPST2 and terminally bled 7 d.p.i. Again, similar levels of ReCD4-Ig (40 ug/mL) were observed in the mice sera of both groups. First, the ability of ReCD4-Ig in the mice sera to neutralize one of the pseudoviruses from the global panel (25710, Tier 2, clade C) was tested using a standard TZM-bl assay (deCamp et al., 2014). It was found that sulfation mediated by IgE-TPST2 significantly enhances the ability of ReCD4-Ig to neutralize this isolate, as evidenced by a right-ward shift of the neutralization curve (FIG. 4B). Specifically, sulfation mediated by IgE-TPST2 decreases IC50 of ReCD4-Ig in neutralizing 25710 from 1.09±0.12 ug/mL to 0.16±0.06 ug/mL (6.8-fold drop) and IC80 from 3.27±0.68 ug/mL to 1.35±0.20 ug/mL (2.4-fold drop). Next, it was evaluated whether ReCD4-Ig can neutralize other isolates from the global panel and tier 3 isolate SIVmac239 and whether IgE-TPST2 mediated sulfation can enhance potency of ReCD4-Ig. It was observed that ReCD4-Ig can neutralize all 13 viruses in the panel with an IC50 less than 5 ug/mL and a mean IC50 of 0.83 ug/mL (FIG. 4C-F). Naïve mice sera, in comparison, did not neutralize any of the virus in the panel at a titer of 1:20. Additionally, ReCD4-Ig in the sera did not non-specifically neutralize murine leukemia virus (MLV) at a titer of 1:8 (or equivalently at an ReCD4-Ig dose of 5 ug/mL). These results validated the remarkable breadth of eCD4-Ig. In addition, sulfation of ReCD4-Ig enhances its potency in neutralizing 8/12 pseudorviruses in the global panel (CE1176, 25710, X2278, TRO, BJOX, X1632, CH119, CNE55) and Mac239 (FIG. 4E). Sulfation exhibits the most drastic effect on the neutralization of CE1176 which exhibits a 10-fold drop in IC50 (0.57±0.27 ug/mL to 0.05±0.02 ug/mL). Overall, IgE-TPST2 mediated sulfation leads to a decrease in the geometric mean of IC50 against the viral panel from 0.83 ug/mL to 0.27 ug/mL. Taken together, these results validated in vivo sulfation of ReCD4-Ig by IgE-TPST2 from a functional standpoint and demonstrated the ability of DNA-encoded enzymes to modulate biological functions of a target protein through post-translational modification.

In Vivo Post-Translational Modification

Experiments were designed using DNA technology as a platform to encode both the eCD4Ig molecule as well as the enzyme IgE-TPST2 to carry out tyrosine sulfation of ReCD4-Ig in vivo. The results presented herein demonstrate a significantly increased potency of the immuneadhesin and provide a unique method for personalized production of such complex molecules.

Importantly, this is the first report of using DNA to encode an enzyme for post-translational modification (PTM) of a target protein for production directly in vivo. As such, these studies support that DNA/EP provides a remarkable platform to modulate the function of protein even after it has been synthesized. For example, modifying the glycosylation of the Fc portion of immunoglobulin can potentially allow for in vivo fine-tuning of effector functions. Afucosylaiton of IgG1 Fc with endoglycosidase/fucosidase, for instance, can potentially enhance antibody-dependent cell-mediated cytotoxicity (ADCC) of the modified antibody; whereas terminal sialyation, in the context of core fucosylation, has been reported to exhibit an opposite effect (Arnold et al., 2007; Li et al., 2017). In the context of vaccine design, post-translational modifications of an antigen can create new epitopes for recognition by the immune system. For example, sialic-acid bearing glycans (at N160, or N156 positions of the HIV envelope) can be recognized by both germline encoded and somatically mutated antibodies in the CAP256.VRC26 ab lineage (Andrabi et al., 2017). Alternatively, post-translational modifications of vaccine antigens can potentially stabilize their conformations in the native states to facilitate mounting of an effective immune response. Tyrosine sulfation of V2 residues on HIV-1 BaL strengthens V2-V3 interactions, improve its recognition by trimer-preferring antibodies PG9, PG16, and PGT145, and reduce its susceptibility to neutralization by anti-V3 antibodies; whereas decrease in its tyrosine sulfation has the opposite effects (Cimbro et al., 2014). Therefore, through enzyme-mediated post-translational modifications of the target proteins encoded by advanced DNA/EP it is likely that modulation of in vivo activity of a variety of important proteins could be approached.

It is demonstrated that a remarkably low dose of 1:1000 p-IgE-TPST2 is required for in vivo sulfation of ReCD4-Ig. This finding was expected since a single molecule of the enzyme should be able to turnover multiple copies of target proteins. Specifically, since TPST2 has a turnover number (k_{cat}) of 5.1×10⁻³s⁻¹ (for mono-sulfated CCR8 peptide) and half-life of a Golgi-resident enzyme is about 20 hours, a single copy of TPST2 enzyme should be able to turnover at least hundreds of copies of ReCD4-Ig (Danan et al., 2010; Strous, 1986). Of note, the dose required to sulfate ReCD4-Ig is much lower for DNA-encoded IgE-TPST2 (1:1000) than AAV-encoded TPST2 (1:4). This implies high efficiency of DNA/EP mediated enzyme delivery and that muscle cells have received separate copies of both p-IgE-TPST2 and p-ReCD4-Ig simultaneously. This is because pulsed electric fields can create transient pores in the plasma membrane, and move polyanionic plasmid DNA directly into the cells, resulting in 100-1000 fold increase in transfection efficiency (Sardesai and Weiner, 2011). In comparison, uptake of AAV-encoded genetic materials (ReCD4-Ig and TPST2) into cells requires clathrin-dependent endocytosis or macropinocytosis (Stoneham et al., 2012; Weinberg et al., 2014), and transduction of muscles cells by both AAV-TPST2 and AAV-eCD4-Ig can occur in a stochastic fashion.

The results also support an approach to target an enzyme to the specific subcellular compartment to maximize its functions. While the efficiency of IgE-TPST2 mediated sulfation appears similar to that of TPST2-mediated sulfation (FIG. 1D), selective targeting of the IgE-TPST2 can potentially reduce cytosolic expression of the enzyme and off-target sulfation. The approach can be further extended to target proteins to other subcellular compartments for therapeutic and investigational purposes. For example, an N-terminal sequence consisting of 10-70 amino acids can target a protein to the mitochondria; dileucine motif DXXLL, or a tyrosine-based motif YXXØ, in the cytoplasmic tail of a transmembrane protein can target proteins to the lysosome; whereas a unit of 5 basic positively charged amino acids on the poplypeptide chain can target a protein to the nucleus (Braulke and Bonifacino, 2009; Lange et al., 2007; Regev-Rudzki et al., 2008).

Finally, it is demonstrated herein that DNA/EP enables robust and long-term in vivo expression of immunoadhesins like ReCD4-Ig. With a single round of injection, a peak expression level of 80-100 ug/mL in mice was observed, with levels that remains above 3 ug/mL for 150 days. This in vivo delivery results in validated breadth and potency of ReCD4-Ig, which can neutralize all isolates from the global panel with an IC50 less than 5 ug/mL and a mean IC50 of 0.27 ug/mL.

In summary, several advances to target an enzyme to the secretory compartment of the cells and the use of DNA/EP for its in vivo expression resulting in significant in vivo potency are described. Importantly, the DNA/EP delivered IgE-TPST2 can significantly enhance potency of eCD4-Ig through in vivo post-translational sulfation likely requires further study as a tool to target HIV infection.

The materials and methods are now described.

Animals

6-8 week old female balb/c and B6.Cg-Foxn1nuJ were obtained from either Charles River (Wilimgton, Mass.) or Jackson laboratory (Bar Harbor, Me.). For DNA delivery, mice were given single intraperitoneal injection of 500 ug of anti-mouse CD40L (clone MR-1, BioXCell) for transient immuno-modulation. They were then given 160 ug (2 injections, left and right TAs) or 320 ug (4 injections, left and right quadriceps, left and right TAs) of DNA co-formulated with 12U of hyaluronidase (Hylenex, catalogue: 18657-117-04) (26,27). 1 minute after injections, IM-EP was performed at each injection site with the Cellectra 3P device (Inovio Pharmaceutical) (Broderick and Humeau, 2015).

DNA Design and Plasmid Synthesis

Protein sequence for ReCD4-Ig was obtained as previously described (Gardner et al., 2015). Protein sequences for human TPST2 and HS3SA were obtained from UniProt (accession numbers: 060704 and Q9Y663). Protein sequence for SIV_{mac239} was obtained from GenBank (accession number M33262). DNA encoding protein sequences were codon and RNA optimized as previously described (Elliott et al., 2017; Patel et al., 2017). The optimized transgenes were synthesized de novo (GenScript, Piscataway, N.J.) and cloned into the pVAX backbone under the control of human CMV promoter and bovine growth hormone poly-adenylation signal. Plasmids that encode HIV envelope gp160 for TRO11, 25710, 398F1, CNE8, X2278, BJOX2000, X1632, CE1176, 246F3, CH119, CE0217 and CNE55 were obtained from NIH-AIDS reagent and amplified at Aldevron LLC (Fargon, N. Dak.).

Cell Lines, Transfection and ReCD4-Ig Purification

HEK293T cells (CRL-3216, ATCC) and TZM-bl cells were maintained in DMEM supplemented with 10% fetal bovine serum and grown at 37° C. and 5% CO2. Expi293F cells were maintained in Expi293 expression medium at 37° C. and 8% CO2. To determine in vitro sulfation of ReCD4-Ig, cells were seeded at a density of 0.5×10⁶ cells/mL in a 6-well plate and transfected with 1.0 μg of p-ReCD4-Ig and varying doses of plasmid encoded enzymes with GeneJammer. 48 hours after transfection, supernatants were collected and centrifuged at 1500 g for 5 minutes to remove cellular debris. Adherent cells were lysed with 1× cell lysis buffer with protease inhibitor cocktail. To obtain ReCD4-Ig standards for quantitative ELISA, Expi 293F cells were plated at a density of 2.5×10⁶ cells/mL in Expi293 expression medium, rested overnight and transfected with p-ReCD4-Ig and Expifectamine™ in OPTI-MEM. Transfection enhancers were added 20 hours after transfection, and supernatant was harvested 5 days after transfection. Magnetic protein G beads (GenScript) were used for purification of ReCD4-Ig, and purity was confirmed with Commassie staining of the SDS-Page gels.

ELISA

For ELISA-based quantification of ReCD4-Ig, MaxiSorp plates were coated with 1 ug/mL of JR-FL gp140 overnight at 4° C. Plates were washed 4 times with Phosphate Buffered Saline+0.1% Tween 20 (PBS-T) and blocked with 10% FBS in PBS for 1 hour at room temperature. Plates were subsequently washed and incubated with serum samples diluted in PBS-T for one hour at room temperature. Plates were washed again and incubated with secondary goat anti-human Fc HRP at 1:5000 dilution for 1 hour. The plates were subsequently developed with SigmaFast OPD for 10 minutes before OD450 measurements were performed with Synergy2 plate reader.

To detect sulfation of ReCD4-Ig in transfection supernatants or sera, MaxiSorp plate were coated at 4° C. overnight with 5 ug/mL JR-FL gp140. Plates were washed and blocked with 10% FBS/PBS for 3 hours at room temperature. Plates were washed, and samples diluted in PBS-T were added for an 1-hour incubation at room temperature. Plates were washed again and incubated with 1:250 dilution of mouse anti-sulfotyrosine antibody (clone 1C-A2, MiliporeSigma) for 1 hour at room temperature. It was discovered that prolonged incubation at this step may increase background. Finally, the plates were washed and incubated with 1:5000 dilution of anti-mouse IgG2a HRP secondary antibody for 1 hour at room temperature. The plates were developed with SigmaFast OPD for 10 minutes and OD450 signals were measured.

Western Blot

For detection of ReCD4-Ig in FIG. 1C, 10 uL of transfection supernatant was loaded onto pre-cast 4-12% Bis-Tris gels under non-reducing condition and transferred to an Immobilon-FL PVDF membrane with wet transfer. ReCD4-Ig was identified with IRDye 800CW goat anti-human IgG (which cross-reacts with Rhesus IgG2 Fc) at 1:10,000 dilution. For detection of sulfated tyrosine in ReCD4-Ig (FIG. 1E), the membrane was incubated overnight with 1:1000 mouse anti-sulfotyrosine (1C-A2) at 4° C. and developed with IRDye 680RD goat anti-mouse IgG. As anti-mouse and anti-human antibodies are conjugated to dyes with different colors, it is possible to visualize ReCD4-Ig and sulfotyrosine bands simultaneously in a single membrane. For detection of IgE-TPST2 expression, mice were sacrificed 7 days after DNA injections/IM-EP. TA muscles were harvested and homogenized in T-PER extraction buffer and protease inhibitor. 50 ug of muscle homogenates were loaded onto 4-12% Bis-Tris gel under reducing condition and transferred to a PVDF membrane with wet transfer. The membrane was incubated overnight with polyclonal rabbit anti-TPST2 antibodies, and monoclonal mouse anti-GAPDH antibody at 4° C. The membrane was subsequently developed with IRDye 680RD goat anti-mouse IgG and IRDye 800CW goat anti-rabbit IgG. All membranes were scanned with Odyssey CLx.

Fluorescence Microscopy

8-well chamber slides (Nunc) were pre-coated with poly-L-lysine solution before HEK293T cells were seeded at a density of 2×10⁵ per well overnight. The cells were then transfected with 1.0 ug of p-ReCD4-Ig and 0.05 ug of p-TPST2, p-IgE-TPST2 or p-ATM-TPST2 with GeneJammer. 48 hours after transfection, the cells were fixed and permeabilized with 4% formaldehyde in PBS and 0.5% Triton-X-100 and blocked with 3% BSA in PBS at room temperature for 1 hour. The cells were then stained overnight at 4° C. with 1:200 dilution of anti-Golgin 97 antibody in 1% BSA/PBS-T, and 1:200 dilution of polyclonal rabbit anti-TPST2 antibody. The cells were then washed with PBS-T and stained with 1:500 dilution of Goat anti-Rabbit Alexa Fluor 594, and Goat anti-Mouse Alexa Fluor 488. For nuclear staining, the cells were incubated with 0.5 ug/mL of DAPI in PBS-T and mounted with cover slips using Prolonged Diamond AntiFade Mountant. Z-stack images were then acquired with Leica TCS SP5 II Scanning Confocal Microscope with a 64× objective. Maximal projections of the Z-stacks, deconvolution, and regions of interest analyses were performed with Leica LASX software to obtain Pearson correlation coefficients to quantify TPST2 and Golgin 97 colocalization.

Ex Vivo Neutralization Assay

Synthesis of HIV Env pseudotyped viruses and TZM-bl assays were performed as previously described (Sarzotti-Kelsoe et al., 2014). Briefly, HEK293 T cells were transfected with 4 ug of plasmid encoding HIV envelope and Bug of plasmid encoding HIV backbone (pSG3Aenv) with GeneJammer. 48 hours after transfection, the supernatants were filtered with Steriflip and stored at −80° C. Pseudoviruses were titrated with a TZM-bl luciferase reporter assay using Britelight Plus to determine a titer that corresponds to at least 150,000 RLU. Mice sera were heat inactivated at 56° C. for 10 minutes for the TZM-bl neutralization assays to determine serum concentration/titer that would result in 50% virus neutralization (IC50).

Statistics

One-way ANOVA analysis and pair-wise T-tests (with Holm-Bonferroni Adjustments in the case of multiple comparisons) were performed with GraphPad Prism 7.0. IC₅₀ values were computed with a non-linear regression model of percentage neutralization versus log (reciprocal serum dilution) using Prism 7.0. P-values less than 0.05 were considered as statistically significant.

### REFERENCES

Andrabi, R., Su, C. Y., Liang, C. H., Shivatare, S. S., Briney, B., Voss, J. E., Nawazi, S. K., Wu, C. Y., Wong, C. H., and Burton, D. R. (2017). Glycans Function as Anchors for Antibodies and Help Drive HIV Broadly Neutralizing Antibody Development. Immunity 47, 524-537 e523.
Arnold, J. N., Wormald, M. R., Sim, R. B., Rudd, P. M., and Dwek, R. A. (2007). The impact of glycosylation on the biological function and structure of human immunoglobulins. Annu Rev Immunol 25, 21-50.
Bradbury, A. F., and Smyth, D. G. (1991). Peptide amidation. Trends Biochem Sci 16, 112-115.
Braulke, T., and Bonifacino, J. S. (2009). Sorting of lysosomal proteins. Biochim Biophys Acta 1793, 605-614.
Broderick, K. E., and Humeau, L. M. (2015). Electroporation-enhanced delivery of nucleic acid vaccines. Expert Rev Vaccines 14, 195-204.
Chen, W., Bardhi, A., Feng, Y., Wang, Y., Qi, Q., Li, W., Zhu, Z., Dyba, M. A., Ying, T., Jiang, S., et al. (2016). Improving the CH1-CK heterodimerization and pharmacokinetics of 4Dm2m, a novel potent CD4-antibody fusion protein against HIV-1. MAbs 8, 761-774.
Cimbro, R., Gallant, T. R., Dolan, M. A., Guzzo, C., Zhang, P., Lin, Y., Miao, H., Van Ryk, D., Arthos, J., Gorshkova, I., et al. (2014). Tyrosine sulfation in the second variable loop (V2) of HIV-1 gp120 stabilizes V2-V3 interaction and modulates neutralization sensitivity. Proc Natl Acad Sci USA 111, 3152-3157.
Danan, L. M., Yu, Z., Ludden, P. J., Jia, W., Moore, K. L., and Leary, J. A. (2010). Catalytic mechanism of Golgi-resident human tyrosylprotein sulfotransferase-2: a mass spectrometry approach. J Am Soc Mass Spectrom 21, 1633-1642.
deCamp, A., Hraber, P., Bailer, R. T., Seaman, M. S., Ochsenbauer, C., Kappes, J., Gottardo, R., Edlefsen, P., Self, S., Tang, H., et al. (2014). Global panel of HIV-1 Env reference strains for standardized assessments of vaccine-elicited neutralizing antibodies. J Virol 88, 2489-2507.
Delorme, E., Lorenzini, T., Giffin, J., Martin, F., Jacobsen, F., Boone, T., and Elliott, S. (1992). Role of glycosylation on the secretion and biological activity of erythropoietin. Biochemistry 31, 9871-9876.
Elliott, S. T. C., Kallewaard, N. L., Benjamin, E., Wachter-Rosati, L., McAuliffe, J. M., Patel, A., Smith, T. R. F., Schultheis, K., Park, D. H., Flingai, S., et al. (2017). DMAb inoculation of synthetic cross reactive antibodies protects against lethal influenza A and B infections. NPJ Vaccines 2, 18.
Gardner, M. R., Kattenhorn, L. M., Kondur, H. R., von Schaewen, M., Dorfman, T., Chiang, J. J., Haworth, K. G., Decker, J. M., Alpert, M. D., Bailey, C. C., et al. (2015). AAV-expressed eCD4-Ig provides durable protection from multiple SHIV challenges. Nature 519, 87-91.
Graf, M., Deml, L., and Wagner, R. (2004). Codon-optimized genes that enable increased heterologous expression in mammalian cells and elicit efficient immune responses in mice after vaccination of naked DNA. Methods Mol Med 94, 197-210.
Harris, R. J. (2005). Heterogeneity of recombinant antibodies: linking structure to function. Dev Biol (Basel) 122, 117-127.
Haryadi, R., Ho, S., Kok, Y. J., Pu, H. X., Zheng, L., Pereira, N. A., Li, B., Bi, X., Goh, L. T., Yang, Y., et al. (2015). Optimization of heavy chain and light chain signal peptides for high level expression of therapeutic antibodies in CHO cells. PLoS One 10, e0116878.
Lange, A., Mills, R. E., Lange, C. J., Stewart, M., Devine, S. E., and Corbett, A. H. (2007). Classical nuclear localization signals: definition, function, and interaction with importin alpha. J Biol Chem 282, 5101-5105.
Li, T., DiLillo, D. J., Bournazos, S., Giddens, J. P., Ravetch, J. V., and Wang, L. X. (2017). Modulating IgG effector function by Fc glycan engineering. Proc Natl Acad Sci USA 114, 3485-3490.
Patel, A., DiGiandomenico, A., Keller, A. E., Smith, T. R. F., Park, D. H., Ramos, S., Schultheis, K., Elliott, S. T. C., Mendoza, J., Broderick, K. E., et al. (2017). An engineered bispecific DNA-encoded IgG antibody protects against Pseudomonas aeruginosa in a pneumonia challenge model. Nat Commun 8, 637.
Prigge, S. T., Mains, R. E., Eipper, B. A., and Amzel, L. M. (2000). New insights into copper monooxygenases and peptide amidation: structure, mechanism and function. Cell Mol Life Sci 57, 1236-1259.
Regev-Rudzki, N., Yogev, O., and Pines, O. (2008). The mitochondrial targeting sequence tilts the balance between mitochondrial and cytosolic dual localization. J Cell Sci 121, 2423-2431.
Ronai, Z., Witt, H., Rickards, O., Destro-Bisol, G., Bradbury, A. R., and Sahin-Toth, M. (2009). A common African polymorphism abolishes tyrosine sulfation of human anionic trypsinogen (PRSS2). Biochem J 418, 155-161.
Sardesai, N. Y., and Weiner, D. B. (2011). Electroporation delivery of DNA vaccines: prospects for success. Curr Opin Immunol 23, 421-429.
Sarzotti-Kelsoe, M., Bailer, R. T., Turk, E., Lin, C. L., Bilska, M., Greene, K. M., Gao, H., Todd, C. A., Ozaki, D. A., Seaman, M. S., et al. (2014). Optimization and validation of the TZM-bl assay for standardized assessments of neutralizing antibodies against HIV-1. J Immunol Methods 409, 131-146.
Son, Y. D., Jeong, Y. T., Park, S. Y., and Kim, J. H. (2011). Enhanced sialylation of recombinant human erythropoietin in Chinese hamster ovary cells by combinatorial engineering of selected genes. Glycobiology 21, 1019-1028.
Stone, S. R., and Hofsteenge, J. (1986). Kinetics of the inhibition of thrombin by hirudin. Biochemistry 25, 4622-4628.
Stoneham, C. A., Hollinshead, M., and Hajitou, A. (2012). Clathrin-mediated endocytosis and subsequent endo-lysosomal trafficking of adeno-associated virus/phage. J Biol Chem 287, 35849-35859.
Strous, G. J. (1986). Golgi and secreted galactosyltransferase. CRC Crit Rev Biochem 21, 119-151.
Sun, Y. M., Jin, D. Y., Camire, R. M., and Stafford, D. W. (2005). Vitamin K epoxide reductase significantly improves carboxylation in a cell line overexpressing factor X. Blood 106, 3811-3815.
Teramoto, T., Fujikawa, Y., Kawaguchi, Y., Kurogi, K., Soejima, M., Adachi, R., Nakanishi, Y., Mishiro-Sato, E., Liu, M. C., Sakakibara, Y., et al. (2013). Crystal structure of human tyrosylprotein sulfotransferase-2 reveals the mechanism of protein tyrosine sulfation reaction. Nat Commun 4, 1572.
Tsang, T. C., Bentley, D. R., Mibashan, R. S., and Giannelli, F. (1988). A factor IX mutation, verified by direct genomic sequencing, causes haemophilia B by a novel mechanism. EMBO J 7, 3009-3015.
Umana, P., Jean-Mairet, J., Moudry, R., Amstutz, H., and Bailey, J. E. (1999). Engineered glycoforms of an antineuroblastoma IgG1 with optimized antibody-dependent cellular cytotoxic activity. Nat Biotechnol 17, 176-180.
Walsh, G., and Jefferis, R. (2006). Post-translational modifications in the context of therapeutic proteins. Nat Biotechnol 24, 1241-1252.
Weinberg, M. S., Nicolson, S., Bhatt, A. P., McLendon, M., Li, C., and Samulski, R. J. (2014). Recombinant adeno-associated virus utilizes cell-specific infectious entry mechanisms. J Virol 88, 12472-12484.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of post-translationally modifying a synthetic protein in a subject, the method comprising administering to the subject a composition comprising a first recombinant nucleic acid sequence encoding the synthetic protein, and a second recombinant nucleic acid sequence encoding a modifier protein, wherein the modifier protein post-translationally modifies the synthetic biologic in the subject.

2. The method of claim 1, wherein the post translational modification is selected from the group consisting of sulfation, acetylation, N-linked glycosylation, myristoylation, palmitoylation, SUMOylation, hydroxylation, methylation, O-linked glycosylation, ubiquitylation, oxidation, and palmitoylation.

3. The method of claim 2, wherein the post translational modification is sulfation and the modifier protein is selected from the group consisting of tyrosylprotein sulfotransferase 1 (TPST1) and TPST2.

4. The method of claim 3, wherein the modifier protein is TPST2.

5. The method of claim 4, wherein TPST2 comprises an IgE leader.

6. The method of claim 5, TPST2 comprises an amino acid sequence at least 90% homologous to SEQ ID NO: 5 or 7.

7. The method of claim 6, wherein the second recombinant nucleic acid sequence comprises a sequence at least 90% homologous to SEQ ID NO: 6 or 8.

8. The method of claim 1, wherein the synthetic protein is an antigen, antibody or immunoadhesin.

9. The method of claim 8, wherein the immunoadhesin is eCD4-Ig.

10. The method of claim 9, wherein eCD4-Ig comprises an amino acid sequence at least 90% homologous to SEQ ID NO:1 or 3.

11. The method of claim 10, wherein the first recombinant nucleic acid sequence comprises a sequence at least 90% homologous to SEQ ID NO:2 or 4.

12. The method of claim 2, wherein the post translational modification is sulfation, the modifier protein is tyrosylprotein sulfotransferase 1 (TPST2), and the synthetic protein is eCD4-Ig, wherein eCD4-Ig is sulfated in the subject.

13. A composition for post-translationally modifying a synthetic protein in a subject comprising:
(a) a first recombinant nucleic acid sequence encoding the synthetic protein, and
(b) a second recombinant nucleic acid sequence encoding a modifier protein.

14. The composition of claim 13, wherein the modifier protein catalyzes a post translational modification (PTM) on the synthetic protein, wherein the PTM is selected from the group consisting of post translational modification is selected from the group consisting of sulfation, acetylation, N-linked glycosylation, myristoylation, palmitoylation, SUMOylation, hydroxylation, methylation, O-linked glycosylation, ubiquitylation, oxidation, and palmitoylation.

15. The composition of claim 14, wherein PTM is sulfation and the modifier protein is selected from the group consisting of tyrosylprotein sulfotransferase 1 (TPST1) and TPST2.

16. The composition of claim 15, wherein the modifier protein is TPST2.

17. The composition of claim 16, wherein TPST2 comprises an IgE leader.

18. The composition of claim 17, TPST2 comprises an amino acid sequence at least 90% homologous to SEQ ID NO: 5 or 7.

19. The composition of claim 18, wherein the second recombinant nucleic acid sequence comprises a sequence at least 90% homologous to SEQ ID NO:6 or 8.

20. The composition of claim 13, wherein the synthetic protein is an antigen, antibody or immunoadhesin.

21. The composition of claim 20, wherein the immunoadhesin is eCD4-Ig.

22. The composition of claim 21, wherein eCD4-Ig comprises an amino acid sequence at least 90% homologous to SEQ ID NO:1 or 3.

23. The composition of claim 22, wherein the first recombinant nucleic acid sequence comprises a sequence at least 90% homologous to SEQ ID NO: 2 or 4.

24. The composition of claim 13, wherein the one or more nucleic acid molecules are engineered to be in an expression vector.

25. The composition of claim 24, further comprising a pharmaceutically acceptable excipient.

26. A method for treating a disease, disorder or infection in a subject in need thereof, the method comprising administering a composition of claim 13 to the subject.

27. The method of claim 26, wherein administering the composition comprises an electroporating step.
